(19) United States
(12) Patent Application Publication
(10) Pub. No.: US 2003/0017530 A1
(43) Pub. Date: Jan. 23, 2003

(54) Heptahelix receptor and its use as leukotriene B4 receptor

(76) Inventor: Christer Owman, Lund (SE)

Correspondence Address: FINNEGAN, HENDERSON, FARABOW, GARRETT &, DUNNER LLP, 1300 I STREET, NW, WASHINGTON, DC, 20005, US

(21) Appl. No.: 09/893,512

(22) Filed: Jun. 29, 2001

Related U.S. Application Data
(63) Continuation of application No. 09/170,069, filed on Oct. 13, 1998, now abandoned.
(60) Provisional application No. 60/061,789, filed on Oct. 14, 1997.
(60) Provisional application No. 60/081,958, filed on Apr. 15, 1998.

(51) Int. Cl. C07K 14/705; C07H 21/04; C12P 21/02; C12N 5/06

(52) U.S. Cl. 435/69.1; 435/325; 435/320.1; 536/23.5; 530/350

(57) ABSTRACT

A full-length cDNA encoding a 352-amino-acid protein contains seven regions of hydrophobic amino acids representing membrane-spanning domains of a heptahelix receptor, tentatively named CMKRL1. It shows nearly 30% overall identity with the c5a anaphylatoxin receptors and similar degree of homology with other chemoattractant receptors. Receptor expression was shown in lymphoid cells and tissues. The receptor and antibodies that recognize the receptor are useful for detecting Burkitt's lymphoma and for lowering leukotriene B4 levels in a mammal.

### CROSS-REFERENCE TO RELATED APPLICATIONS

This regular U.S. patent application claims the benefit of U.S. provisional patent application No. 60/061,789, filed Oct. 14, 1997, and U.S. provisional patent application No. 60/081,958, filed Apr. 15, 1998, the entire disclosures of which are relied upon and incorporated herein by reference.

### BACKGROUND OF THE INVENTION

This invention relates to a new chemoattractant receptor and its use. More particularly, this invention relates to cDNA encoding a novel heptahelix-like receptor expressed in lymphoid cells and tissues. This invention also relates to a method of detecting lymphomas. In addition, since the receptor is a receptor for leukotriene B4, this invention also relates to methods aimed at influencing the trafficking of inflammatory cells, at treating diseases in which leukotriene signaling is abnormal (for example, asthma, rheumatoid arthritis, psoriasis, AIDS, and other immunodeficiency conditions), and at lowering active leukotriene levels in mammals.

The superfamily of G-protein-coupled membrane receptors receives signals that show great variety in chemical structure and originate from a large number of sources (Watson and Arkinstall, 1994). Recent rapid development in the understanding of the molecular mechanisms underlying the functions of these receptors is to a large extent based on the cloning and structural analysis. of cDNA or genes encoding the receptors, not least the receptors receiving signals from neurotransmitter amines and peptides. Such work was pioneered by the groups of Lefkowitz (cf. Dixon et al., 1986), Numa (cf. Kubo et al., 1986), and Nakanishi (cf. Masu et al., 1987).

Because of the continuing interest in chemoattractant receptors, there exists a need in the art for the identification of new receptors and the elucidation of their structure so that the function of these receptors in humans can be determined. In particular, there exists a need in the art for sequence information on such receptors, including DNA and amino acid sequences, to enable the isolation and characterization of particular receptors as well as structurally related receptors.

The identification of receptors associated with pathogenic conditions would be particularly advantageous in assays for the identification of these conditions in susceptible individuals.

### SUMMARY OF THE INVENTION

Accordingly, this invention aids in fulfilling these needs in the art. More particularly, this invention provides a B-cell lymphoblast derived heptahelix receptor that is strongly expressed in lymphoid cells and tissues, including leukocytes, lymph nodes, thymus, spleen, and bone marrow.

More particularly, the present invention provides isolated heptahelix receptors and DNA sequences encoding these heptahelix receptors. Such DNA sequences include (a) cDNA clones having a nucleotide sequence derived from the coding region of a native heptahelix receptor gene of the invention; (b) DNA sequences that are capable of hybridization to the cDNA clones of (a) under moderately stringent conditions and which encode biologically active heptahelix receptor molecules; and (c) DNA sequences that are degenerate as a result of the genetic code to the DNA. sequences defined in (a) and (b) and which encode biologically active heptahelix receptor molecules.

The present invention also provides recombinant expression vectors comprising the DNA sequences defined above, recombinant heptahelix receptor molecules produced using the recombinant expression vectors, and processes for producing the recombinant heptahelix receptor molecules using the expression vectors.

The present invention further provides isolated or purified protein compositions comprising heptahelix receptor of the invention.

In one embodiment of the invention, a heptahelix receptor of the invention has the following amino acid sequence and corresponding nucleotide sequences:

CLONING OF NOVEL HUMAN CHEMOATTRACTANT RECEPTOR

| | | |
|---|---|---|
| −308 | acctgctacttgaaggccacacccagcc | −361 |
| −360 | tctcactcccttaccttcctcttcctctctcactgctccttcctggtctcttctcatctggccccacctctaaggcgtcctcctgccttctgggttgccctggaaaatagactacccccc | −241 |
| −240 | ctcctagtgaagggagtgggtaggggtttcagccccaccctcaggaagatgcgtcttccctgtcctctgctctgtggtacttcctctctggctgatttagcaaacagcacctagacctgg | −121 |
| −120 | gccaggcctttggcagtgggacagacccagggataggctacaccaccctgccctgaccctgggattggcatcagcttccaaccagttcctgccaaagcttgtaaggtcctcccgacggcc | −1 |
| | [symbol] I | |
| 1 | M X T T S S A A P P S L G V E F I S \overline{L L A I I L L S V A L A V G L P G S S T V V} | 40 |
| 1 | atgaacactacatcttctgcagcacccccctcactaggtgtagagttcatctctctgctggctatcatcctgctgtcagtggxgxtggctgtggggcttcccggcaacagctttgtggtg | 120 |
| | II | |
| 41 | \overline{W S} I L K R M Q K R S V T A \overline{L M V L M L A L A D L A V L L T A P F F L} N F L A Q | 80 |
| 121 | tggagtatcctgaaaaggatgcagaagcgctctgtcactgccctgatggtgctgaacctggccctggccgacctggccgtattgctcactgctccctttttccttcacttcctggcccaa | 240 |
| | III | |
| 81 | G T W S F G L A G C R L \overline{C M Y V C G V S M T A S V L L I T A K S L D} R S L A V A | 120 |
| 241 | ggcacctggagttttggactggctggctggttgccgcctgtgccactatgtctgcggagtcagcatgtacgccagtgtcctgcctatcacggtcatgagtctagaccgctcactggcggtggcc | |
| | IV | |
| 121 | A P F V S Q K L A T K A N A R R \overline{V L A G I M V L S P L L A T P V L A Y A T} V V P | 160 |
| 361 | cgcccctttgtgtcccagaagctacgcaccaaggcgatggcccggcgggtgctggcaggtatctgggtgttgtcctttctgctggccacacccgtcctcgcgtaccggacagtagtgccc | 480 |
| | [symbol] V | |
| 161 | W X T M M S L C F P R Y P S E G H R \overline{A F K L I F X A V T G F L L P F L A V V A S} | 200 |
| 481 | tggaaaacgaacatgagcctgtgcttcccgcggtaccccagcgaagggcaccgggccttccatctaatcttcgaggctgtcacgggcttcctgctgcccttcctggctgtggtggccagc | 600 |
| | VI | |
| 201 | Y S D I G R R L Q A R X R R F R R S R R T G R L \overline{V V L I I L T F A A F W L P Y M V V }240 | |
| 601 | tactcggacatagggcgccggctacaggcccggcgcttccgccgcagccgccgcaccggccgcctggtggtgctcatcatcctgaccttcgccgccttctggctgccctaccacgtggtg | 720 |
| | VII | |
| 241 | \overline{M L} A E A R R A L A C Q A A G L G L V G K R L S L A R X V L \overline{I A L A F L S S S V } | 280 |
| 721 | aacctggctgaggcccgccgcgcgctggccggccaggccgccgggttagggctcgtggggaagcggctgagcctggcccgtaacgtgcttatcgcactcgccttcctgagcagcagcgtg | 840 |
| 281 | \overline{W P V L Y A C A G G G} L L R S A G V G F V A K L L E G T G S K A S S T R R G G S | 320 |
| 841 | aaccccgtgctgtacgcgtgcgccggcggcggcctgctgcgtttggcgggcgtgggctttgtcgccaagctgctggagggcacgggctccgaggcgtccagcacgccgcgggggcagc | 960 |
| 321 | L G Q T A R S G P A A L X F G P S K S L T A S S F L K L N E L M | 352 |
| 961 | gctgggccagaccgctaggagcggccccgccgctctggagcccggcccttccgagagcctcactgcctccagccctctcaagttaaacgaactgaactaggcctggtggaaggaggcgcat | 1080 |
| 1081 | tttcctcctggcagaatgctagctctgagccagttcagtacctggaggagcaggggcgtggagggcgtggagggcgtgggagcgtgggaggcgggagtggagtggaagaagagggag | 1200 |
| 1201 | aggtggagcaaagtgagggccgagtgagagcgtgctccagcctggctcccacaggcagctttaaccattaaaactgaagtctga | 1283 |

SEQ ID NO:1.

The heptahelix receptor compositions of the invention are useful in diagnostic assays for heptahelix receptor, as well as in raising antibodies toheptahelix receptor for use in diagnosis and therapy.

This invention also provides a method of detecting Burkitt's lymphoma, wherein the method comprises providing human cells, contacting the cells with an antibody of the invention, and detecting immunological complex containing the antibody.

Because the heptahelix receptor of the invention is a leukotriene receptor for leukotriene B4, the invention is useful for detecting LTB4, for example, by immunoassay for hepzahelix polypeptides of the invention or by detection of all or part of the polynucleotides encoding the receptor of the invention, such Has by hybridization assays or amplification reactions.

In addition, the heptahelix receptor compositions can be used directly in therapy to bind or scavenge chemoattractants, thereby providing a means for regulating the immune activities of chemoattractants.

More particularly, this invention provides a method for lowering the levels of active leukotriene B4 in a mammal in need thereof, which comprises administering to the mammal a leukotriene B4-lowering amount of a leukotriene B4 receptor comprising the sequence of amino acids of SEQ ID NO:l.

In another embodiment, this invention provides a method for assaying a ligand or an antagonist or agonist for said ligand.

The method comprises:

(A) providing a heptahelix receptor of the invention or a fragment thereof comprising a binding domain for the ligand, antagonist, or agonist;

(B) incubating the receptor with a test sample suspected to contain the ligand, antagonist, or agonist; and

(C) detecting binding between the receptor and the ligand, antagonist, or agonist.

The receptor can be in an external cell membrane of a host cell transfected or transduced with DNA encoding the receptor. In addition, binding can be detected by intracellular calcium level in the host cell.

The heptahelix receptor of the invention is a novel chemoattractant receptor, provisionally named CMKRL1, which has turned out to be the first leukotriene (LT) receptor. Following this identification, the provisional designation CMKRL1 is changed into BLTR, which is the leukotriene B4 receptor according to the pharmacologic nomenclature of NC-IUPHAR. Binding assays using tritiated LTB₄ and isolated membranes from COS-7 cells, transiently transfected with cDNA encoding this receptor, yielded a linear Scatchhard plot showing expression of only a single, high-affinity receptor population with a mean K_{d} of 2.1 nM and Bₘₐₓ of 17.0 pmoles/mg protein. Sham-transfected cells exhibited no specific binding. LTB₄ elicited concentration-dependent increases in intracellular calcium measured with Fura-2 in individual CHO cells stably expressing CMKRL1. No response was seen with sham-transfected control cells or in calcium-free medium, which suggests that calcium mainly originates from extracellular sources. The LTB₄-induced cellular calcium increment was blocked in the presence of a monoclonal antibody raised against a synthetic peptide corresponding to the extracellular tail of CMKRL1 and capable of visualizing the receptor by fluorescence immunocytochemistry. Taken together the analyses show that LTB₄ is the endogenous ligand for CMKRL1.

### BRIEF DESCRIPTION OF THE DRAWINGS

This invention will be more fully described with reference to the drawings in which:

FIG. 1 shows the nucleotide sequence of the coding region of Lyme21-9 and the deduced amino acid sequence of the corresponding human CMKRL1 receptor. The first nucleotide and amino acid residue of the translation start site are designated as position 1. The putative transmembrane segments TMI-TMVII are indicated by solid lines; the extension of each segment is estimated on the basis of the hydrophobicity profile and sequence alignment of other heptahelix receptors. Potential glycosylation sites are indicated with arrowheads.

FIG. 2 is the hydropathy pattern of the deduced amino acid sequence of Lyme21-9 according to the algorithm of Kyte and Doolittle (1982). The horizontal figures show the amino acid numbers. Regions (indicated positive) containing the maximally hydrophobic amino acid residues (over a window of nine in the standard GCG program) are numbered (I-VII)

FIG. 3 depicts the alignment of the complete amino acid sequences for eight human chemotactic receptors together with the amino acid sequence deduced from the presently cloned cDNA (Lyme21-9) showing the high degree of similarity (shaded areas), not least within the transmembrane regions. The homology presentation was done with the SeqVu (version 1) mode in the GCG program. The scaling system used is described by Riek et al. (1995).

FIG. 4 depicts a dendrogram (the horizontal distances to the branching points corresponding to the relative degree of sequence identities) based on the eight human chemoattractant receptors listed in FIG. 3 as well as the Duffy antigen, together with the currently cloned Lyme21-9, showing similarities in the evolutionary pattern. For comparison, the dendrogram also includes a selection of “unrelated” human receptors belonging to the rhodopsin-type family: the amine receptors histamine H1, muscarinic M1, dopamine D₂, adrenergic β₂ and the peptide receptors neuromedin K, substance P, and substance K. Full amino acid sequences were used in the multiple alignments and the dendrogram and they were performed with the PileUp software in the GCG program.

FIG. 5 depicts fluorescence photomicrographs with examples of FISH mapping of the gene corresponding to CMKRL1.

FIG. 5A shows fluorescent signals on one human chromosome.

FIG. 5B shows the same mitotic figures stained with DAPI to identify chromosome 14. Original magnification was x1300.

FIG. 6 shows a diagram of FISH/DAPI mapping results. Each dot represents the double fluorescent signals detected on chromosome 14 (images from 10 photographs).

FIG. 7 shows results of a Northern blot hybridization of mRNA (2 μg poly(A)+ RNA per lane) from 28 human tissues and cells with the Lyme21-9 cDNA probe. Constantly appearing hybridizing transcripts are approximately 5 and 7.5 kb (RNA molecular size markers are shown on both sides), appearing alone or in pairs, in addition to a 3-kb transcript, appearing alone in some tissues. Yet smaller bands are seen in bone marrow and with lower signal intensity also in leukocytes. The cancer cell lines are promyelocytic leukemia HL-60, HeLa cells S3, chronic myelogenous leukemia K-562, lymphoblastic leukemia MOLT-4, Burkitt lymphoma Raji, colorectal adenocarcinoma SW480, lung carcinoma A549, and melanoma G361.

FIG. 8 is a saturation and Scatchard (inset) analysis of [³H]LTB₄ binding to membranes prepared from COS-7 cells transfected with Lyme21-9 cDNA to express CMKRL1. Results shown are the mean value of 3 experiments performed in triplicate.

FIG. 9 is a specific binding of [³H]LTB₄ to membranes prepared from COS-7 cells transfected with the CMKRL1 expression plasmid, Lyme21-9, as a function of membrane concentration. Various (protein) concentrations of membranes were incubated with 2 nM tritiated LTB₄ for 60 min at 4° C., with or without 1 μM unlabeled LTB₄. Results are mean values of triplicate wells.

FIG. 10 shows the action of 4 different concentrations of LTB₄ (figures given are final concentrations in the test well) on intracellular calcium levels in CHO cells stably transfected to express CMKRL1 and preloaded with Fura-2 for monitoring of intracellular calcium concentration (fluorescence ratios). The lower curve illustrates the effect of the highest LTB₄ concentration tested on sham-transfected control cells. Values are means ±SEM (n).

FIG. 11 shows the effect of 1000 nM (final concentration in test well) of LTB₄ on intracellular calcium levels in CHO cells transfected to express CMKRL1 (and preloaded with Fura-2 for monitoring of calcium fluorescence ratios). Effects of LTB₄ are compared in calcium (Ca) containing and calcium-free cell media. Values are means ÷SEM (n).

FIG. 12 reports the results of fluorescence immunocytochemistry of a monoclonal antibody (mAb) raised against a synthetic peptide corresponding to the first 15 amino acid residues in the extracellular tail of CMKRL1 showing (a) finely-granular fluorescence in the periphery of CHO cells stably expressing CMKRL1, and (b) absence of fluorescence in sham-transfected control cells. Magnification: 500×.

FIG. 13 shows the effect of monoclonal antibody (rnAb) that is illustrated in FIG. 12 on the cellular calcium influx induced by 1000 nM LTB, in CHO cells, which stably express CMKRL1. Values are means ±SEM (n).

### DETAILED DESCRIPTION OF THE INVENTION

The native, mature full-length human heptahelix receptor of the invention is a 352 amino acid protein having a molecular weight of about 43 kilodaltons (kDa) as deduced from the number of amino acid residues in the molecule. This heptahelix receptor has been given the designation “CMKRL1”. CMKRL1 has been found to be a receptor for leukotriene B4, hereinafter named BLTR.

Specifically, binding assays and functional experiments conclusively show that CMKRL1 is the receptor for leukotriene B₄ (LTB,). This potent inflammatory chemoattractant derivative of arachidonic acid is a product of the 5-lipoxygenase pathway and is formed from LTA₄ by a specific hydrolase. The name leukotriene derives from the notion that leukotrienes can be produced by leukocytes and that a conjugated triene forms a common structural entity. They are members of the group of eicosanoids, which also contain, e.g., prostaglandins and thromboxane. The leukotrienes mediate their biological actions via high affinity stereoselective membrane-bound receptors.

More particularly, the leukotrienes have been known as well-characterized chemical entities since the late seventies (8), and it has been known that their biological actions are mediated through high-affinity stereoselective membrane receptors. Nevertheless, no sequence information has been available at either the amino acid or nucleotide level (9) until the cDNA encoding the corresponding receptor was cloned two decades later (1,4). Despite the longstanding absence of structural information, the LTB₄ receptor has, nevertheless, been extensively investigated as a pharmacological entity (9) and is known under the BLT receptor designation (10).

LTB₄ is a proinflammatory mediator, which is a potent activator principally of polymorphonuclear leukocytes (PMN), but also of related myeloid cells and mast cells. LTB₄ is one of a number of chemoattractant substances, including the synthetic peptide formyl-methionyl-leucyl-phenylalanine (FMLP), complement component C5a, interleukin 8 and platelet-activating factor, which have similar biological effects and together regulate leukocyte function through interaction with independent receptors. LTB₄ induces chemotaxis, chemokinesis, and aggregation causing the migration ofneutrophils to the site of inflammation where these cells degranulate resulting in the release of lysosomal enzymes, in addition to other antibacterial and microbicidal agents, as well as superoxide production as aspects of the host defense to pathogens (Ford-Hutchinson et al., 1980). In addition, LTB₄ also promotes adherence of neutrophils to endothelial cell walls, and in concert with other mediators can thereby amplify the inflammatory response. Most of the effects of LTB₄ have been studied using PMN, which are also the major site of synthesis for LTB₄, although similar effects on monocvzes and eosinophils have been reported. In addition, LTB₄ may play a role in the immune system through modulation of both T- and B-lymphocyte function (Metfters, 1995).

It has now been discovered that tritiated LTB₄ binds with high affinity to a single receptor population in CQS-7 cells transfected with cloned cDNA encoding CMKRL1 (1) owing to the marked structural homology with members of the chemoattractant receptor group. Moreover, LTB₄ activates transfected CHO cells stably expressing CMKRL1 receptor message resulting in a concentration-dependent increase in the cellular flux of calcium. The effect is blocked by a mAb obtained by immunizing mice with a synthetic peptide corresponding to the N-terminus of the deduced receptor protein. No binding or activation is seen with LTB₄ in sham-transfected control cells. This shows that LTB₄ is an endogenous functional ligand for CMKRL1, which is the first cloned leukotriene receptor. Subsequently cloned cDNA and receptor sequences are identical (2,4), whereas clone R2 obtained from a human genomic library (7) deviates in the 51 end, which led to the identification of a different methionine initiation site in the proposed coding sequence.

The affinity constant presently obtained in the binding of tritiated LTB₄ to transfected COS-7 cell membranes is close to the figure from guinea-pig eosinophil membranes (11), but higher than the value from retinoic-acid differentiated HL-60 cells or COS-7 cells (4). Differences in biological activity of the receptor may be related to how the receptor is expressed in the membrane of various cell types. This could, among other things, allow for a varying degree of dimerization that might affect receptor affinity (12). In this context it is notable that higher molecular weight receptor species have been reported for another chemoattractant receptor, namely C5a (13).

The interaction of LTB₄ with the cloned BLTR is counteracted by receptor antagonist as well as by various eicosanoids (4), and it is also blocked by mAb described hereinafter against a synthetic N-terminal receptor peptide. The N-terminus is the most immunogenic domain of the seven-transmembrane receptor (14), and synthetic peptides corresponding to this region have been used successfully for production of mAbs recognizing other related receptors, such as IL-8R (14) and CCR3 (15). This domain seems to be the primary ligand.binding site for, e.g., the chemoattractant receptor, C5a (16). It may be a mechanism through which a mAb recognizing this region blocks BLTR receptor function by directly competing with the ligand binding, by occupying space indirectly preventing the access of LTB₄ to other (secondary) binding domains, or by inducing conformational changes in the receptor molecule leading to its temporary inactivation.

The G-protein mediated intracellular signaling activated by LTB₄ in transfected CHO cells seems to involve both an increase in intracellular calcium levels mediated by G_{q}, and an inhibition of adenylyl cyclase activity through G₁ (4). The blockade of the intracellular calcium augmentation obtained during stimulation with LTB₄ in calcium-free medium indicates that the increased level of the ion in the CHO cells is derived mainly from extracellular sources, probably through activation of receptor-operated channels in the cell membrane. It is well recognized that signal transduction from a particular receptor may vary with the type of cell owing to the diversity in the effector system (17, 18) and its availability in any given cell expressing that particular receptor.

LTB₄-induced PMN activation in vivo causes neutrophil invasion and accumulation in the lungs, peritoneal cavity, skin and eyes of experimental animals. BLTR activation, therefore, has been proposed as an important event in pathologies where infiltrating neutrophils are present, for example inflammatory bowel disease (IBD) and psoriasis. (Metters, 1995).

Thus, in general terms, this invention relates to the discovery of a new heptahelix receptor. As used herein, the term “heptahelix receptor” refers to proteins and polypeptides having amino acid sequences that are substantially similar to this native, mature, mammalian heptahelix receptor amino acid sequence, and which are biologically active, as defined below, in that they are capable of binding chemoattractant molecules or transducing a biological signal initiated by a chemoattractant or other ligand binding to a cell as CMKRL1 is able to do, or cross-reacting with anti-heptahelix receptor antibodies raised against heptahelix receptor from natural (i.e., nonrecorbinant) sources. As used throughout the specification, the term “mature” means a protein expressed in a form lacking a leader sequence as may be present in full-length transcripts of a native gene.

The term “heptahelix receptor” includes, but is not limited to, analogs or subunits of native proteins having seven membrane-spanning or hydrophobic regions and which exhibit at least some biological activity in common with CMKRL1. For example, soluble heptahelix receptor constructs, which possess the seven hydrophobic regions (and are secreted from the cell), and retain the ability to bind chembattractants are deemed to be heptahelix receptors of the invention.

In the absence of any species designation, heptahelix receptor refers generically to mammalian heptahelix receptor. Similarly, in the absence of any specific designation for deletion mutants, the term heptahelix receptor means all forms of heptahelix receptor, including mutants and analogs that possess heptahelix receptor biological activity.

“Soluble heptahelix receptor” as used in the context of the present invention refers to proteins and polypeptides, or substantially equivalent analogs, having an amino acid sequence corresponding to all or part of the heptahelix receptor, and which are biologically active in that they bind to chemoattractants and other related ligands. Soluble heptahelix receptors include polypeptides that vary from those sequences by one or more substitutions, deletions, or additions, and which retain the ability to bind chemoattractant or inhibit chemoattractant signal transduction activity via cell surface bound heptahelix receptor proteins. Inhibition of chemoattractant signal transduction activity can be determined by transfecting cells with recombinant heptahelix receptor DNAs to obtain recombinant receptor expression. The cells can then be contacted with chemoattractant or other ligand and the resulting metabolic or signal transduction effects examined. If an effect results that is attributable to the action of the chemoattract or other ligand, then the recombinant receptor has signal transduction activity. Procedures for determining whether a polypeptide has signal transduction activity include measurement of adenyl cyclase activity and intracellular calcium measurements.

The group of chemoattractant or “chemokine-like” substances as used in the context of the present invention comprises both the “classical” chemoattractants and the chemokines. Examples of the “classical” chemoattractants, as one having ordinary skill in the art would appreciate, include but are not limited to LTB4, PAF, fMLP, C3a, and C5a. In addition, all subtypes of the chemokines, i.e., alpha, beta, etc., are within the context of the present invention.

The term “isolated” or “purified”, as used in the context of this invention to define the purity of heptahelix receptor protein or protein compositions means that the protein or protein composition is separated from its native environment. In one embodiment, the heptahelix receptor can be substantially free of other proteins of natural or endogenous origin. In a preferred embodiment, the receptor can contain less than about 1% by mass of protein contaminants residual of its native environment or production processes. Such compositions, however, can contain other proteins added as stabilizers, excipients, or cotherapeutics.

The term “substantially similar” when used to define either amino acid or nucleic acid sequences means that a particular subject sequence, for example, a mutant sequence, varies from a reference sequence by one or more substitutions, deletions, or additions, the net effect of which is to retain biological activity of the heptahelix receptor as may be determined, for example, in a chemoattractant assay for heptahelix receptor binding. Alternatively, nucleic acid subunits and analogs are “substantially similar” to the specific DNA sequences disclosed Wherein if: (a) the DNA sequence is derived from the coding region of native, mammalian heptahelix receptor gene of the invention; (b) the DNA sequence is capable of hybridization to DNA sequences of (a) and which encode biologically active heptahelix receptor molecules; or DNA sequences that are degenerate as a result of the genetic code to the DNA sequences defined in (a) or (b) and which encode biologically active heptahelix receptor.

The heptahelix receptor of the invention can be obtained Ifrom natural sources or by recombinant techniques using eukaryotic or procaryotic host systems. “Recombinant” as used herein means that a protein is derived from recombinant (e.g., microbial or mammalian) expression systems. “Microbial” refers to recombinant proteins made in bacterial or fungal (e.g., yeast) expression systems. As a product, “recombinant microbial” defines a protein produced in a microbial expression system that is essentially free of native endogenous substances. Protein expressed in most bacterial cultures, e.g., E. coli, will be free of glycan. Protein expressed in yeast may have a glycosylation pattern different from that expressed in mammalian cells.

“Biologically active” as used throughout this specification as a characteristic of heptahelix receptors of the invention means that a particular molecule shares sufficient amino acid sequence similarity with embodiments of the present invention to be capable of binding chemoattractant or other ligand, transmitting a stimulus to a cell, for example, as a component of a hybrid receptor construct, or cross-reacting with anti-heptahelix receptor antibodies raised against heptahelix receptor from natural (i.e., nonrecombinant) sources.

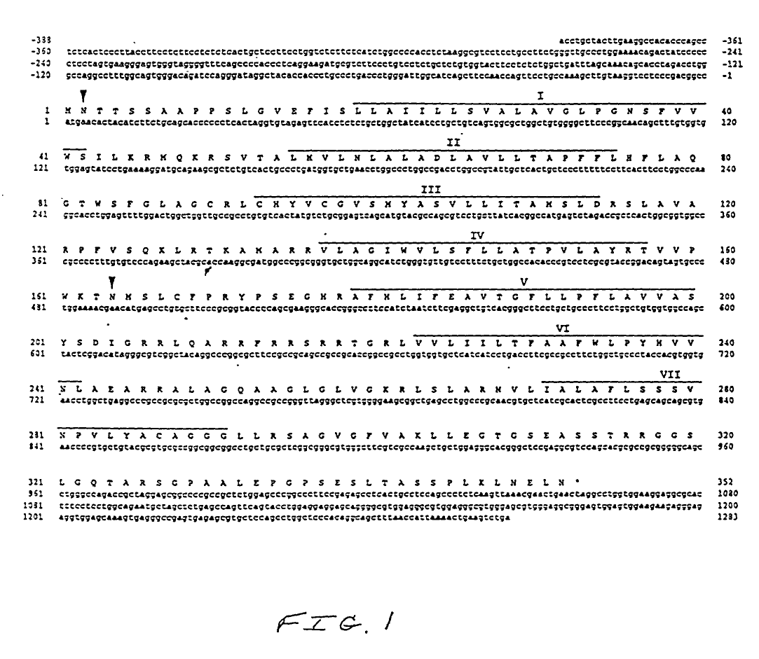

“Isolated DNA sequence” refers to a DNA polymer in the form of a separate fragment or as a component of a larger DNA construct, which has been derived from DNA isolated at least once. Preferably, the heptahelix receptor is in substantially pure form, i.e., free of contaminating endogenous materials and in a quantity or concentration enabling identification, manipulation, and recovery of the sequence and its component nucleotide sequences by standard biochemical methods. Such sequences are preferably provided in the form of an open reading frame uninterrupted by internal, nontranslated sequences, or introns, which are typically present in eukaryotic genes. Such a sequence is shown in FIG. 1 for the preferred heptahelix receptor, CMKRL1. Genomic DNA containing the relevant sequences can also be used as a source of coding sequences. The genomic location of such DNA is described hereinafter. Sequences of non-translated DNA may be present 5′ or 3′ from the open reading frame, where the same do not interfere with manipulation or expression of the coding regions.

“Nucleotide sequence” refers to a heteropolymer of deoxyribonucleotides. DNA sequences encoding the heptahelix receptors of this invention can be assembled from DNA fragments, such as short oligonucleotide linkers, or from a series of oligonucleotides, or by amplification reactions to provide a synthetic gene that is capable of being expressed in a recombinant transcriptional unit. With this background, the characteristics of one embodiment of the invention will now be described in detail.

A full-length cDNA (Lyme21-9) encoding a novel putative seven-transmembrane (7TM) domain receptor, named CMKRL1, was identified in a human B-cell lymphoblast library by screening with degenerate primers and radiolabeled oligonucleotide probes corresponding to neurotransmitter receptors. Highest expression of receptor message was found in leukocytes, lymph nodes, thymus, and bone marrow. Northern blot hybridization with a specific, full-length cDNA probe showed, even under high stringency conditions, several bands with sizes varying between approximately 7.5 and 1.5 kb. Only the smallest bands corresponded to the size of the cloned cDNA. There could be several reasons for this, given that the hybridization signal is specific. There may be splice variants of the message and/or a varying expression of different entities in the course of cell maturation. Indeed, several potential splice sites, identified by, e.g., the base combination CAGG (cf. Mattaj and Seraphin, 1992), can be seen in the currently cloned cDNA sequence. The cDNA may have been synthesized primarily from the short mRNA individuals, or even from truncated forms, in the construction of the cDNA library (Okayama et al., 1987).

While many cloned G-protein-linked receptors belong to the neurotransmitter receptor category, only relatively few are specifically associated with the immune system (e.g., Murphy, 1994; Watson and Arkinstall, 1994; Kunkel et al., 1995). Several novel receptor DNA sequences that may belong to this functional category based solely on predicted amino acid similarity have been cloned-during the past few years, though the ligands involved have not yet been identified. Members of this receptor family are the BLRs, expressed in Burkitt lymphoma and in lymphocyte cell lineages (Dobner et al., 1992).

Another heptahelix type Burkitt lymphoma receptor from the same B-lymphoblast library used in the present study was cloned (Owman, 1996). The peptide sequence deduced from that cDNA shows, however, only approximately 22% identity with the corresponding sequence of Lyme21-9, and while ubiquitously distributed, there is no expression in peripheral blood leukocytes, in contrast to the situation for Lyme21-9.

Recently, another 7TM receptor expressed in leukocyzes, LESTR, was cloned from a monocyte library (Loetscher et al., 1994) and was found to be the human equivalent of the bovine brain boLCR1, which was mistakenly thought to be a neuropeptide Y (NPY) receptor (Rimland et al, 1991). A cDNA sequence w-th more than 90% amino acid identity to the bovine sequence has been cloned from human lung and kidney libraries, and the corresponding gene was found to reside in chromosome 2 (Herzog et al., 1993). Strong attention has recently been given to these receptors because they seem to be an entry-cofactor for HIV-1 (Feng et al., 1996) in CD4-positive target cells.

Another lymphoid-specific receptor, EBIL, originally identified as an Epstein-Barr-induced cDNA (Birkenbach et al., 1993), has turned out to be expressed also in normal lymphoid tissues; the corresponding gene is located in human chromosome 17 (Schweickart et al., 1994). The IL-8 receptor genes are clustered on human chromosome 2 (Ahuja et al., 1992).

The present Lyme21-9 gene was found to have yet another localization-on chromosome 14. The chemokine-like human receptor gene, R2, has recently been mapped to this chromosome as well (Raport et al., 1996). Thus, there seems to be no common denominator in this context for a clue to receptor identification. Indeed, a search in the Genome Data Base did not provide any further clues about possible ligands or any disease of interest mapped in human chromosomes to the particular region in which the CMKRL1 gene is localized.

Because of the similarity to chemoattractant receptors and the high expression in lymphoid tissues including leukocytes, the currently cloned receptor was designated CMKRL1 in accordance with the Genome Database Nomenclature rules, which stands for chemoattractant receptor-like 1.

Murphy (1994) has listed five consensus features that appear to identify chemoattractant receptors as a distinct subfamily among the heptahelix receptors. Thus, their sequences are similar in length, approximately 350 amino acid residues (CMKRL1 is 352 residues long), which places them among the smallest in the receptor superfamily. In addition, they show around 30% overall identity (the same as CMKRL1) in their peptide sequence. The third intracellular loop is short (16-22 amino acid residues; the deduced peptide of CMKRL1 has 21 residues) and is enriched in basic amino acids. In contrast, the N-terminal segments are usually acidic in the chemoattractant receptors, like the currently cloned receptor. Finally, a common characteristic of the receptors is expression of their messages in leukocytes.

More particularly, using PCR with degenerate primers and screening of a human B-cell lymphoblast cDNA library, a full-length cDNA encoding a 352 amino acid protein was isolated. It contains seven regions of hydrophobic amino acids probably representing the membrane-spanning regions of a novel heptahelix receptor, named CMKRL1. It showed over 30% overall identity with the C5a anaphylatoxin receptors. The coding region consists of 1056 bp corresponding to 352 amino acid residues and giving an approximate molecular weight of 43 kDa. Northern blot analysis showed hybridizing transcripts in spleen, thymus, and lymph nodes, as well as in bone marrow and peripheral blood leukocytes. Message was also found in lymphoid tumor cell lines. Chromosome mapping with FISH/DAPI technique showed the corresponding gene to reside on human chromosome 14qll.2-ql2. Furthermore, stably transfected mammalian cells expressing high levels of corresponding receptor RNA were analyzed for changes in cAMP concentration and cellular calcium fluxes.

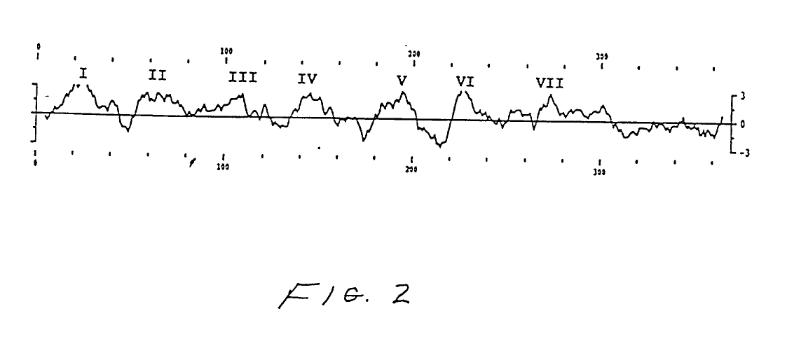

FIG. 1 shows the nucleotide sequence of this clone (Lyme21-9) and the amino acid sequence deduced from the coding region of the cDNA; The hydrophobicity profile (FIG. 2) of the peptide sequence shows the presence of seven segments probably forming the membrane-spanning regions of the receptor, which, for reasons given further below, was named CMKRL1.

The predicted amino acid sequence also possesses other features common to the superfamily of G-protein-coupled receptors: (i) consensus sequences for N-linked glycosylation (Asn-X-Ser/Thr, where X is any amino acid) near the amino terminus (Asn-2) and in the second extracellular loop (Asn-164); (ii) a conserved cysteine residue in each of the first two extracellular loops (Cys-90 and Cys-168), providing possibilities to form a disulfide bond that stabilizes the functional protein structure; (iii) proline residues in all transmembrane regions (except TMIII) thought to induce flexibility within the helix formations; and (iv) a carboxyl terminus with several serine and threonine residues (10/4) that could serve as substrate for serine/threonine protein kinases. The predicted third intracellular loop, thought to be involved in the coupling to G-proteins and varying most in length among the different receptors, consists of only 21 amino acid residues.

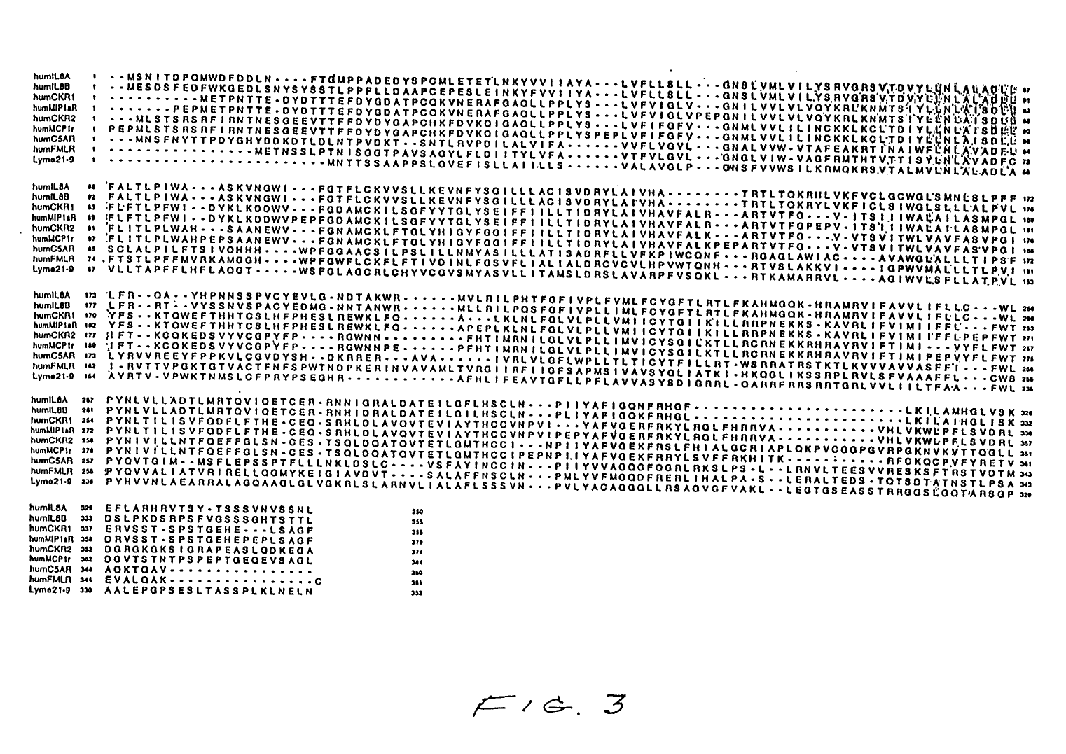

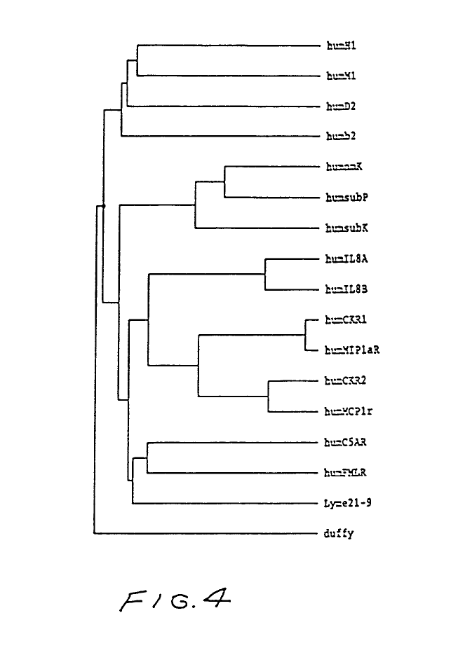

Sequence comparison with cloned receptors within the G-protein-linked superfamily showed most similarity with the subfamily of chemoattractant leukocyte receptors (FIG. 3), particularly the “classical” chemoattractants, C5a and N-formyl-methionyl-leucyl-phenylalanine (fMLP) (FIG. 4). For example, there is (in the GCG/fasta matrix score) an approximately 30% overall sequence identity with the human C5a anaphylatoxin receptor (Gerard and Gerard, 1991) and 28% identity with the fMLP receptor (Boulay et.al., 1990; Thomas at al., 1990). Taken as a group together with the Lyme21-9 receptor cDNA clone (FIG. 3), there is a particularly high degree of consensus in the GN-LVVLV sequence motif in the TMI region and the LLNLA—DLLF—TLP-W motif within TMII.

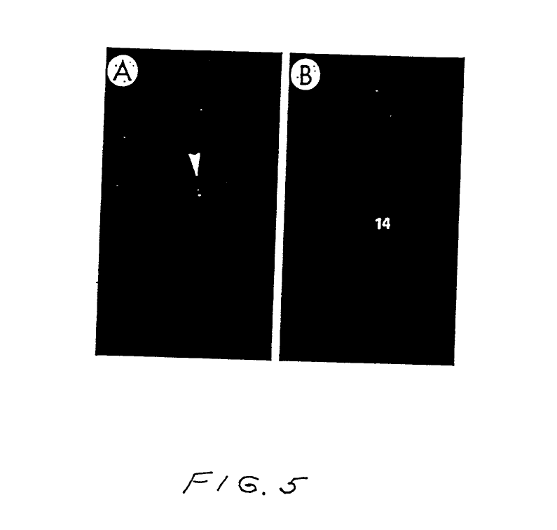

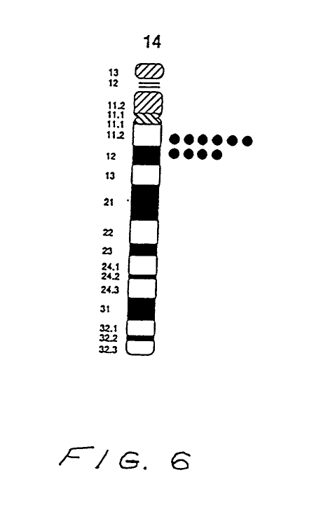

The hybridization efficiency in the chromosome mapping with FISH was 68% (i.e., among 100 checked mitotic FIGS. 68 showed signals on one pair of the chromosome). Based on the DAPI banding, an assignment was obtained between the fluorescent signal from the Lyme21-9 DNA probe and the long arm of chromosome 14 (FIG. 5). The detailed position was further determined as a summation from 10 photomicrographs, indicating localization of the corresponding gene to region q11.2-q12 (FIG. 6).

The tissue distribution of the receptor in terms of message established in Northern blot hybridization was analyzed with the use of a full-length, gene-specific cDNA probe at high stringency.

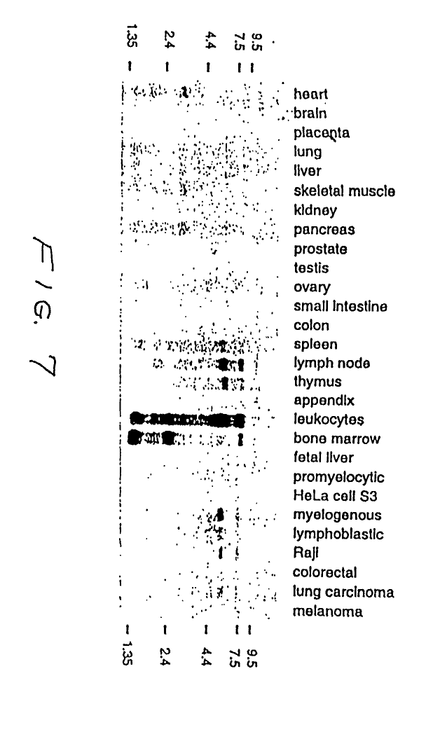

As shown in FIG. 7, the hybridization revealed two primary transcripts of slightly more than 5 and 7.5 kb in spleen, thymus, lymph nodes, bone marrow (large transcript predominating), and peripheral blood leukocytes (small transcript predominating) with the above-mentioned order of autoradiographic signal intensity.

The two transcripts, alone or together, were expressed in some human cancer cell lines (FIG. 7): promyelocytic leukemia HL-60, lymphoblastic leukemia MOLT-4, Burkitt lymphoma Raji, lung carcinoma A549, and chronic myelogenous leukemia K-562. Some tissues, including skeletal muscle, pancreas, and heart, showed only one, relatively weak, hybridizing band that was of smaller size, about 3 kb (FIG. 7).

Two smaller bands appeared in the bone marrow, and they were seen at a lower signal intensity also in peripheral leukocytes. The tissue distribution of the message was corroborated in RT-PCR experiments on normal human tissue.

Hybridization with a structurally related but different cDNA probe, the Burkitt lymphoma receptor clone, showed an entirely different hybridization pattern.

Since the receptor is structurally similar to the chemoattractant receptors, particularly the “classical” ones, but also the C—C or β-chemokine receptors, it was named CMKRL1 (which stands for “chemoattractant receptor-like l,” in conformity with the Genome Database Nomenclature rules).

Both L-VIP reporter cells and CHO cells were transfected with the Lyme21-9 plasmid, and permanently expressing clones of both cell lines were successfully established as revealed by positive bands appearing in Northern blot hybridization with the full-length cDNA (Lyme21-9) probe.

It will be understood that the heptahelix receptor of the invention and its corresponding DNA template is not limited to the CMKRL1 receptor and Lyme21-9 clones just described. For example, additional cDNA clones can be isolated from cDNA libraries of other mammalian species by cross-species hybridization. Mammalian heptahelix receptor of the present invention includes by way of example, primate, human, murine, ,canine, feline, bovine, ovine, equine, and porcine heptahelix receptor. Mammalian heptahelix receptors can be obtained by cross-species hybridization using a single-stranded cDNA derived from the human heptahelix receptor DNA sequence as a hybridization probe to isolate heptahelix receptor cDNAs from Imammalian cDNA libraries. For use in hybridization, DNA encoding heptahelix receptor can be covalently labeled with a detectable substance, such as a fluorescent group, a radioactive atom, or a achemiluminescent group, by methods well known to those skilled in ithe art. Such probes can also be used for in vitro diagnosis of particular conditions.

Alternative mRNA constructs, which can be attributed to different mRNA splicing events following transcription and which share large regions of identity or similarity with the heptahelix receptors claimed herein, are considered to be within the scope of the present invention:

Derivatives of heptahelix receptor within the scope of the invention include various structural forms of the primary protein, which retain biological activity. Due to the presence of ionizable amino and carboxyl groups, for example, a heptahelix receptor protein can be in the form of acidic or basic salts, or can be in neutral form. Individual amino acid residues can also be modified by oxidation or reduction.

The primary amino acid structure can be modified by forming covalent or aggregative conjugates with other chemical moieties, such as glycosyl groups, lipids, phosphate, acetyl groups, and the like, or by creating amino acid sequence mutants. Covalent derivatives can be prepared by linking particular functional groups to heptahelix receptor amino acid side chains or at the N- or C-termini. Other derivatives of heptahelix receptor within the scope of this invention include covalent or aggregative conjugates of heptahelix receptor or its fragments with other proteins or polypeptides, such as by synthesis in recombinant culture as N-terminal or C-terminal fusions. For example, the conjugated peptide can be a signal (or leader) polypeptide sequence at the N-terminal region of the protein, which co-translationally or post-translationally directs transfer of the protein from its site of synthesis to its site of function inside or outside of the cell membrane or wall (e.g., the yeast a-factor leader). Heptahelix receptor protein fusions can comprise peptides added to facilitate purification or identification of heptahelix receptor (e.g., poly-His).

Heptahelix receptor derivatives can also be used as immunogens, reagents in receptor-based immunoassays, or as binding agents for affinity purification procedures of chemoattractants or other binding ligands. Heptahelix receptor derivatives can also be obtained by cross-linking agents, such as M-maleimidobenzoyl succinimide ester and N-hydroxysuccinimide, at cysteine and lysine residues. Heptahelix receptor proteins can also be covalently bound through reactive side groups to various insoluble substrates, such as cyanogen bromide-activated, bisoxirane-activated, carbonyldiimidazole-activated, or tosyl-activated agarose structures, or by adsorbing to polyolefin surfaces (with or without glutaraldehyde cross-linking). Once bound to a substrate, heptahelix receptor can be used to selectively bind (for purposes of assay or purification) anti-heptahelix receptor antibodies or chemoattractant.

The present invention also includes heptahelix receptor, with or without associated native-pattern glycosylation.

Heptahelix receptor expressed in yeast or mammalian expression systems can be similar or slightly different in molecular weight and glycosylation pattern than the native molecules, depending upon the expression system. Expression of heptahelix receptor DNAs in bacteria, such as E. coli, provides non-glycosylated molecules. Functional mutant analogs of mammalian heptahelix receptor having inactivated N-glycosylation sites can be produced by oligonucleotide synthesis and ligation or by site-specific mutagenesis techniques. These analog proteins can be produced in a homogeneous, reduced-carbohydrate form in good yield using yeast expression systems.

Heptahelix receptor derivatives can also be obtained by mutations of heptahelix receptor or its subunits. A heptahelix receptor mutant, as referred to herein, is a polypeptide homologous to heptahelix receptor, but which has an amino acid sequence different from native heptahelix receptor because of a deletion, insertion, or substitution.

Bioequivalent analogs of heptahelix receptor proteins can be constructed by, for example, making various substitutions of residues or sequences, or deleting terminal or internal residues or sequences, or deleting terminal or internal residues or sequences not needed for biological activity. For example, cysteine residues can be deleted (e.g., Cys¹⁷⁸) or replaced with other amino-acids to prevent formation of unnecessary or incorrect intramolecular disulfide bridges upon renaturation. Generally, substitutions should be made conservatively; i.e., the most preferred substitute amino acids are those having physiochemical characteristics resembling those of the residue to be replaced. Similarly, when a deletion or insertion strategy is adopted, the potential effect of the deletion or insertion on biological activity should be considered. Substantially similar polypeptide sequences, as defined above, generally comprise a like number of amino acids.

In order to preserve the biological activity of heptahelix receptors, deletions and substitutions will preferably result in homologously or conservatively substituted sequences, meaning that a given residue is replaced by a biologically similar residue. Examples of conservative substitutions include substitution of one aliphatic residue for another, such as Ile, Val, Leu, or Ala for one another, or substitution of one polar residue for another, such as between Lys and Arg; Glu and Asp; or Gln and Asn. Other such conservative substitutions, for example, substitutions of entire regions having similar hydrophobicity characteristics, are well known.

Mutations in nucleotide sequences constructed for expression of analog heptahelix receptor must, of course, preserve the reading frame phase of the coding sequences and preferably will not create complementary regions that could hybridize to produce secondary mRNA structures, such as loops or hairpins, which would adversely affect translation of the receptor mRNA. Although a mutation site may be predetermined, it is not necessary that the nature of the mutation per se be predetermined. For example, in order to select for optimum characteristics of mutants at a given site, random mutagenesis may be conducted at the target codon and the expressed heptahelix receptor mutants screened for the desired activity.

Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene having particular codons altered according to the substitution, deletion, or insertion required.

The present invention provides recombinant expression vectors to amplify or express DNA encoding heptahelix receptor. Recombinant expression vectors are replicable DNA constructs that have synthetic or cDNA-derived DNA fragments encoding mammalian heptahelix receptor or bioequivalent analogs operably linked to suitable transcriptional or translational regulatory elements derived from mammalian, microbial, viral, or insect genes. A transcriptional unit generally comprises an assembly of (1) a genetic element or elements having a regulatory role in gene expression, for example, transcriptional promoters or enhancers; (2) a structural or coding sequence, which is transcribed into mRNA and translated into the heptahelix receptor; and (3) appropriate transcription and translation initiation and termination sequences. Such regulatory elements can include an operator sequence to control transcription and a sequence encoding suitable mRNA ribosomal binding sites. The ability to replicate in a host, usually conferred by an origin of replication, and a selection gene to facilitate recognition of transformants can additionally be incorporated.

DNA regions are operably linked when they are functionally related to each other. For example, DNA for a signal peptide (secretory leader) is operably linked to DNA for a polypeptide if it is expressed as a precursor, which participates in the secretion of the polypeptide. A promoter is operably linked to a coding sequence if it controls the transcription of the sequence. A ribosome binding site is operably linked to a coding sequence if it is positioned so as to permit translation. Generally, operably linked means contiguous and, in the case of secretory leaders, contiguous and in reading frame.

Structural elements intended for use in yeast expression systems preferably include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

DNA sequences encoding mammalian heptahelix receptors, which are to be expressed in a microorganism, preferably contain no introns that could prematurely terminate transcription of DNA into mRNA; however, premature termination of transcription may be desirable, for example, where it would result in mutants having advantageous C-terminal truncations. Due to code degeneracy, there can be considerable variation in nucleotide sequences encoding the same amino acid sequence. Other embodiments include sequences capable of hybridizing to the sequences of the provided cDNA under moderately stringent conditions and other sequences hybridizing or degenerate to those that encode biologically active heptahelix receptor polypeptides.

Recombinant heptahelix receptor DNA can be expressed or amplified in a recombinant expression system comprising a substantially homogenous monoculture of suitable host microorganisms, for example, bacteria such as E. coli, or yeast such as S. cerevisiae, which have stably integrated (by transduction or transfection) a recombinant transcriptional unit into chromosomal DNA or carry the recombinant transcriptional unit as a component of a resident plasmid. Generally, cells constituting the system are the progeny of a single ancestral transformant. Recombinant expression systems as defined herein will express heterologous protein upon induction of the regulatory elements linked to the DNA sequence or synthetic gene to be expressed.

Transformed host cells are cells that have been transduced or transfected with heptahelix receptor vectors constructed using recombinant DNA techniques. Transformed host cells ordinarily express heptahelix receptor, but host cells transformed for purposes of cloning or amplifying heptahelix receptor DNA do not need to express heptahelix receptor. Expressed heptahelix receptor will be deposited in the cell membrane or secreted into the culture supernatant, depending on the heptahelix receptor DNA selected. Suitable host cells for expression of heptahelix receptor include prokaryotes, yeast, or higher eukaryotic cells under the control of appropriate promoters. Prokaryotes include gram-negative or gram-positive organisms, for example E. coli or bacilli. Higher eukaryotic cells include established cell lines of mammalian origin. Cell-free translation systems can also be employed to produce heptahelix receptor using RNAs derived from the DNA constructs of the present invention. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference.

Promoters commonly used in recombinant microbial expression vectors include the β-lactamase (penicillinase) and lactose promoter system (Chang et al., Nature 275:615, 1978; and Goeddel et al., Nature 281:544, 1979), the tryptophan (tp) promoter system (Goeddel et al., Nucl. Acids Res. 8:40576, 1980; and EPA 36,776), and tac promoter (Maniatis, Molecular Cloning: A Laboratory Manual, Cold Spring Harbor Laboratory, p. 412, 1982).

Recombinant heptahelix receptor proteins can also be expressed in yeast hosts, preferably from the Saccharomyces species, such as S. cerevisiae. Yeast of other genera, such as Pichia or Kluyveromyces, can also be employed. Yeast vectors will generally contain an origin of replication from the 2μ yeast plasmid or an autonomously replicating sequence (ARS), promoter, DNA encoding heptahelix receptor, sequences for polyadenylation, Itranscription, termination, and a selection gene.

Suitable promoter sequences in yeast vectors include the promoters for metallothionein, 3-phosphoglycerate kinase (Hitzeman et al. J. Biol. Chem. 255:2073, 1980), or other glycolytic enzymes (Hess et al. J. Adv. Enzyme Reg. 7:149, 1968); and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, phosphoglucose isomerase, and glucokinase.

Various mammalian or insect cell culture systems are also advantageously employed to express recombinant protein. Expression of recombinant proteins in mammalian cells is particularly preferred because such proteins are generally correctly folded, appropriately modified, and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), HeLa, and BHK cell lines. Mammalian expression vectors can comprise nontranscribed elements, such as an origin of replication, a suitable promoter, and an enhancer linked to the gene to be expressed, and other 5′ or 3′ nontranslated sequences, such as ribosome binding sites and Itranscriptional termination sequences. Baculovirus systems for production of heterologous proteins in insect cells are reviewed by Luckow and Summers, Bio/Technology 6:47 (1988).

The transcriptional and translational control sequences in expression vectors to be used in transforming vertebrate cells can be provided by viral sources. For example, commonly used promoters and enhancers are derived from polyoma, adenovirus 2, Simian Virus 40 (SV40), and human cytomegalovirus. DNA sequences derived from the SV40 viral genome, for example, SV40 origin, early and late promoter, enhancer, splice, and polyadenylation sites, can be used to provide the other genetic elements required for expression of a heterologous DNA sequence.

Purified mammalian heptahelix receptors or analogs can be prepared by culturing suitable host/vector systems to express the recombinant translation products of the DNAs of the present invention, which are then purified from culture media or cell extracts. For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. For example, a suitable affinity matrix can comprise a heptahelix or lectin or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose, or other types commonly employed in protein purification.

Finally, one or more reverse-phase high performance liquid chromatography (RP-HPLC) steps employing hydrophobic RP-HPLC media, e.g., silica gel having pendant methyl or other aliphatic groups, can be employed to further purify a heptahelix receptor-containing composition. Some or all of the foregoing purification steps, in various combinations, can also be employed to provide a homogeneous heptahelix receptor.

Recombinant heptahelix receptor produced in bacterial culture can be isolated by initial extraction from cell pellets, followed by one or more concentration, salting-out, aqueous ion exchange, or size exclusion chromatography steps. Finally, high performance liquid chromatography (HPLC) can be employed for final purification steps. Microbial cells employed in expression of recombinant the receptor can be disrupted by any convenient method, including freeze-thaw cycling, sonication, mechanical disruption, or use of cell lysing agents.

Heptahelix receptors synthesized in recombinant culture are characterized by the presence of non-human cell components, including proteins, in amounts and of a character which depend upon the purification steps taken to recover the receptor from the culture. These components ordinarily will be of yeast, prokaryotic, or non-human higher eukaryotic origin and preferably are present in innocuous contaminant quantities, on the order of less than about 1 percent by weight. Further, recombinant cell culture enables the production of heptahelix receptor free of proteins that may be normally associated with receptor as it is found in nature in its species of origin, e.g., in cells, cell exudates, or body fluids.

The heptahelix receptor of the invention is a novel chemoattractant, which has turned out to be the first leukotriene (LT) receptor. Accordingly, the present invention provides methods of using therapeutic compositions comprising an effective amount of soluble heptahelix receptor and a suitable diluent or carrier in the treatment of a mammal, including primate, human, murine, canine, feline, bovine, ovine, equine, or porcine species. The invention includes methods for modulating heptahelix receptor-dependent responses in humans comprising administering an effective amount of soluble heptahelix receptor to a patient. Appropriate dosages can be determined in trials. The amount and frequency of administration will depend, of course, on such factors as the nature and severity of the indication being treated, the desired response, the condition of the patient, and so forth.

More particularly, phagocytes important in immunology are polymorphonuclear leukocytes (e.g. neutrophils) and mononuclear phagocytes (e.g. monocytes and macrophages). Phagocyte hypofunction is a cause of recurrent pyogenic infection. To combat pyogenic infection, neutrophils and monocytes respond to chemotactic factors by moving toward the source of infection, where they ingest microorganisms and kill them.

More particularly, a main function of polymorphonuclear leukocytes and monocytes is to kill bacteria and other infectious agents by phagocytosis. The first stage in the ingestion and digestion of a particulate substance by these cells involves the process of bringing the cells and the particles together, usually through chemotaxis. This response is an essential part of host defense against infection. The extensive migration and activity of these cells are manifested by inflammation at the site of injury or invasion of the host.

It has been shown that LTB₄ induces chemotaxis by neutrophils. The heptahelix receptor of the invention is capable of modulating the chemoattractant effect of LTB₄ chemotaxis. This can be demonstrated by carrying out an assay for neutrophil chemotaxis using LTB₄ and the heptahelix receptor of the invention. By the use of the heptahelix receptor of the invention, it is possible to inhibit chemotaxis of granulocytes, monocytes, and macrophages. Thus, the receptor of the invention is capable of modulating directional movement of cells. This invention makes it possible to inhibit inflammation associated with cell chemotaxis. The receptor can be administered to a patient to inhibit the effects of chemotactic factors of bacterial or viral origin, or components of plasma activation systems, or factors elaborated by cells of the immune system.

Leukocyte response to an acute inflammatory stimulus involves a complex series of events, including adherence to endothelium near the stimulus. Inhibition of leukocyte adherence can be expected to reduce the degree of inflammation seen in conditions, such a septic shock and adult respiratory distress syndrome. LTB₄ promotes adherence of polymorphonuclear leukocytes (PMN), such as neutrophils, to endothelial cell walls. Since the heptahelix receptor of this invention is a receptor for LTB, the receptor can effectively block such adherence.

Polymorphonuclear leukocyte (PMN) adherence to nylon can be readily demonstrated.

Purified PMN cells can be incubated with a lipopolysaccharide-stimulated mononuclear leukocyte conditioned medium containing LTB₄. PMN adherence to nylon can be determined without the receptor of this invention, and then with the receptor of this invention. The compounds employed in the process of this invention are effective in blocking adherence of leukocytes and thereby aiding in reducing the degree of inflammation.

Mature phagocytes are in a metabolically dormant state. It is currently believed that recognition of certain objects and substances by phagocytes, such as the attachment of an ingestible particle to the cell surface, changes this situation, and the cell enters a stage of increased metabolic activity, which is referred to as metabolic or respiratory burst. The transition is associated with a series of characteristic changes, including the production of a superoxide anion. LTB₄ is capable of producing a similar effect. In addition to its significance for phagocytic function related to inactivation of ingested microbes, activation of oxygen metabolism is a useful indirect marker for the ingestion process per se. It would be desirable to be able to modulate the effect of LTB₄ on respiratory burst.

Quantitative methods for direct measurement of hydrogen peroxide and superoxide anions released into the medium are currently available. The heptahelix receptor employed in this invention is capable of modulating respiratory burst in stimulated polymorphonuclear leukocytes (PMN) as determined using these methods. The heptahelix receptor used in the process of this invention is capable of reducing superoxide production and modulating respiratory burst in phagocytes, such as polymorphonuclear leukocytes and monocytes, and thereby aid in reducing the degree of inflammation.

During ingestion, granules in the cytoplasm of the cell fuse with the membrane of a vacuole that was formed around the foreign substance. The granules discharge their contents into the vacuole. Some of this material ends up in the medium surrounding the phagocyte. Since the granules disappear during this process, it is called degranulatidn. The granule contents include hydrolytic enzymes, lysozyme, bactericidal proteins, and, in the neutrophil, myleoperoxidase.

Degranulation can be assessed by measuring the rate of appearance of granule-associated enzymes in the extracellular medium. In the case of polymorphonuclear leukocytes (PMN), degranulation can be assayed by determining release of lysozyme. LTB₄ induces the release of lysosomal enzymes. The heptahelix receptor employed in the process of this invention is capable of modulating the release of lysozyme from stimulated PMN and thereby aid in reducing the degree of inflammation.

In summary, the heptahelix receptor of the invention is capable of modulating the effects of leukotrienes, such as LTB₄, on polymorphonuclear leukocytes and mononuclear phagocytes. The receptor is capable of inhibiting cell chemotaxis. In addition, the receptor can block adherence of cells. The compounds can decrease oxidative damage to host tissues by phagocytes as evidenced by modulation of respiratory burst in cells stimulated by LTB₄. Finally, the receptor can modulate the effects of LTB₄ on degranulation in stimulated phagocytes. These effects are suggestive of clinical effectiveness in at least the following areas and conditions.

Among the conditions that can be treated or alleviated by the inhibition of LTB₄ are: sepsis, septic shock, endotoxic shock, gram negative sepsis, toxic shock syndrome, adult respiratory distress, cachexia secondary to AIDS, rheumatoid arthritis, and gouty arthritis.

By reference to the specific cause of the disease condition, the more generic term “trauma” can be used. The term “trauma” refers broadly to cellular attack by foreign bodies, such as microorganisms, particulate matter, chemical agents, and the like. Included among physical injuries are mechanical injuries, such as those caused by contact with sources of electrical injuries, such as those caused by contact with sources of electrical potential; and radiation damage caused, for example, by prolonged, extensive exposure to infrared, ultraviolet, or ionizing radiations.

LTB₄ is also characteristically a product of blood phagocytes. The leukotrienes bind to specific receptors on smooth muscle cells and cause prolonged bronchoconstriction. The receptor of this invention is useful for alleviating this condition, including asthmatic bronchoconstriction.

A preferred embodiment of the invention will now be described in still greater detail in the following Examples. These examples are offered by way of illustration, and not by way of limitation.

### EXAMPLE 1

### Isolation of cDNA Clone

A cDNA library of a human B-cell lymphoblast cell line (GM03299; NIGMS Human Genetic Mutant Cell Repository, Camden, N.J.) was constructed from poly(A)⁺-selected RNA in the pcD/SP6/T7 cloning and expression vector (Morel et al., 1992), a derivative of Okayama-Berg's pcD vector (Okayama et al., 1987). The library contained 7.5×10⁶ recombinants. Plasmid preparation was prepared by cesium chloride-ethidium bromide banding (Sambrook et al., 1989) and used as template (1 μg) in PCR (Mullis and Faloona, 1987) attempting to amplify a DNA stretch between the putative TMII and TMVI of G-protein-coupled receptors. The sense primer was a 27-mer oligonucleotide with 250-fold degeneracy (5′-A(T)TCCTGGTG(C)A(T)G(A)CCTT(G)GCT(A)G(T)TGGCC(T)GAC-3′); the antisense primer was a 29-mer oligonucleotide with 128-fold degeneracy (5′-AT(G)GA(T)AGA(T)AGGGCAGCCAGCAGAC(G)C(G)G(A)T(C)GAA-3′). The primers were used in 1 μM concentrations together with Tag polymerase (Geneamp; Perkin-Elmer Cetus). Forty cycles of 96° C. for 45 s (denaturation), 55° C. for 4 min (annealing), and 72° C. for 4 min (extension) were carried out, followed by a final extension at 72° C. for 15 min. The products were analyzed on a 3% NuSieve genetic technology-grade agarose gel (FMC BioProducts). Three bands between 500 and 700 bp in size were excised and blunted with T4 polymerase, and terminal phosphates were added with T4 polynucleotide kinase (New England Biolabs). The fragments were subcloned into the HincII site of the M13mp18 vector and sequenced according to Sanger's dideoxynucleotide termination method. Several sequences exhibited homology with the G-protein-coupled superfamily. Sequence information from one insert (hLym10) was utilized to obtain a full-length cDNA clone.

### EXAMPLE 2

### Screening of cDNA Library

On the basis of sequence stretches in the PCR clone corresponding to the putative first extracellular and third intracellular loops, two 48-bp oligonucleotides were synthesized, one designated Lym5, 5′-ACACAGGAGGCAACCAGCCAGTCCAAAACTCCAGGTGCCTTGGGCCAG-3′, and the other Lym6, 5′-GATCGGTGCCAGCACCCGCCGGGCCATCGCCTTGGTGCGTAGCTTCTG-3′. They were labeled with [γ-³²P]ATP (5000 Ci/mmol, Amersham) and used in combination as probes to screen pools of recombinants prepared from consecutive dilutions (Bonner et al., 1987) of the human B-cell lymphoblast cDNA library. Hybridization of Southern blots was performed in 3× SSC (0.45 M NaCl, 0.05 M sodium citrate, pH 7.0) at 60° C., and the filters were washed in 1× SSC at the same temperature. A positive band of 1.7 kb in size was followed until a single clone (designated Lyme211-9) was obtained. Overlapping restriction fragments were subcloned into M13 phage vectors for sequencing of both cDNA strands. Sequence analysis and comparisons were performed with Genetics Computer Group software (University of Wisconsin) and with GenBank as well as with the GeneWorks program from IntelliGenetics (Mountain View, Calif.). Hydrophobicity tests of the deduced amino acid sequence were carried out according to Kyte and Doolittle (1982). Chromosome mapping results were evaluated in the Genome Data Base (GDB 6.0) and the NCBI database (Online Mendelian Inheritance in Man; OMIM).

### EXAMPLE 3

### Chromosome Mapping

The procedures followed were those of Heng and Tsui (1993, 1994). Lymphocytes isolated from human cord blood were cultured in α-MEM medium supplemented with 10% fetal calf serum and phytohemagglutinin at 37° C. for 68-72 h. The cultures were treated with 5-bromo-2′-deoxyuridine (0.18 mg/ml, Sigma) to synchronize the cell population. The synchronized cells were washed three times with serum-free medium to release the block and recultured at 37° C. for 6 h in a-MEM with thymidine (2.5 μg/ml, Sigma). Cells were harvested and suspensions dropped on slides using standard procedures including hypotonic treatment, fixation, and air-drying.

The probe used consisted of the entire plasmid construct of pcD vector including the Lyme21-9 cDNA insert. It was biotinylated with DATP at 15° C. for 2 h using the BioNick labeling kit (BRL).

For detection with fluorescence in situ hybridization (FISH) the slides were baked at 55° C. for 1 h. After RNase A treatment the cell spreads were denatured in 70% formamide in 2× SSC for 1 min at 70° C. followed by dehydration with ethanol. The biotinylated DNA probe was denatured at 75° C. for 5 min in a hybridization mix consisting of 50% formamide and 10% dextran sulfate and human cot I DNA (BRL), and prehybridized for 15 min at 37° C. The probe mix was loaded on the denatured slides and hybridized overnight. The slides were washed and the hybridization signal was recorded in a fluorescence microscope, photographed, and amplified as described (Heng and Tsui, 1994). The assignment of FISH mapping data with a chromosomal banding pattern following staining with 41-6-diamidino-2-phenylindole (DAPI) was achieved by superimposing photographic images according to Heng and Tsui (1993).

### EXAMPLE 4

### Cell Transfection

Intact plasmid DNA (10-25 μg) was used to transfect two cell lines: Chinese hamster ovary cells (CHO-K1 cells; CCL61, American Type Culture Collection, Rockville, Md.) and mouse LVIP2.0Zc cells (Konig et al., 1991), which contain a stably integrated fusion gene, pVIP2.OZ plasmid (Riabowol et al., 1988), consisting of the Escherichia coli LacZ gene under the transcriptional control of a fragment derived from the human vasoactive intestinal polypeptide (VIP) gene. The calcium phosphate precipitation method (Chen and Okayama, 1987) was used, and the pcDₙₑₒ vector was cotransfected to allow for selection with the neomycin analog, G-418 (Geneticin, 500 pg/ml), which was started 72 h after the transfection and onward. The L cells were already maintained in the presence of 25 μg/ml hygromycin as a selectable marker for phyg, a plasmid encoding hygromycin B phospho-transferase (Sugden et al., 1985), which had originally been cotransfected along with the VIP reporter construct. Monoclonal lines expressing the Lyme21-9 cDNA were obtained by limiting dilution. Expression of the corresponding mRNA was verified by Northern blot hybridization with the same probes (Lym5 and Lym6) as used in the original library screening. Sham-transfected cells were used as controls.

### EXAMPLE 5

### Measurement of Adenylyl Cyclase Activity

Changes in cAMP levels of the whole transfected cells were assayed using two approaches. One was a semiquantitative screening of candidate receptor ligands utilizing the mouse L cells. These were seeded into 96-well microtiter plates at 5×10⁴ to 10×10⁴ cells per well in 100 ml medium and incubated for an additional 24 h. Then another 100 ml medium was added containing the phosphodiesterase inhibitor, isobutyl methyl-xanthine (IBMX), and the test ligand. Ligands were assayed for inhibition of adenylyl cyclase in the presence of 1 μM forskolin. The chromogenic substrate, o-nitrophenyl-b-D-galactopyranoside (ONPG, Research Organics), was added and any color change was measured in a plate reader (Molecular Devices) at 405 nm wavelength.

In the other method, cAMP accumulation was determined quantitatively in radioimmunoassays using ¹²⁵I-succinyl-cAMP as tracer (Brooker et al., 1979). CHO cells were seeded in 24-well clusters at a density of 10⁶ cells per well and grown to confluence. The growth medium was replaced with 250 ml medium containing the test agents and 1 μM IBMX. Forskolin (1 μM) was added to allow for recording of any inhibitory responses. The reaction was stopped after 5 min with 250 ml ice-cold solution containing 0.1 N HCl and 1 mM CaCl₂. Radioactivity was measured in a gamma counter with 85% counting efficiency.

### EXAMPLE 6

### Intracellular Calcium Measurements

Transfected and nontransfected cells were seeded on 30-mm-diameter glass coverslips and grown for 1-2 days to 40-95% confluence. They were loaded with 2 μM Fura-2 in 500 μl culture medium for 30 min at 37° C. in the presence of 2.5 mM probenecid. After three washes in extracellular medium buffer (135 mM NaCl, 4.6 mM KCl, 1.2 mM MgCl₂, 1.5 mM CaCl₂, 11 mM glucose, 11 mM Hepes, pH 7.4) the coverslips were mounted in a custom-made perfusion chamber on the stage of a Nikon inverted fluorescence microscope fitted to a photometer (Photon Technology International, Model D104). Measurements were carried out in extracellular medium buffer at room temperature, and the test agonists were added with a micropipette. Fluorescence images were obtained by alternate excitation at 340 and 380 nm, and the emitted light was measured at 510 nm. The slit was adjusted to allow for measurements from a single cell.

### EXAMPLE 7

### Tissue Distribution of Receptor mRNA

Northern blot hybridization was carried out according to the manufacturer's protocol (except for the use of 60° C. washing temperature at 0.1× SSC) on premade nitrocellulose multiple tissue filters from Clontech (MTN Blot I and II, HCCL) covering 28 human tissues and cell lines. The hybridization probe consisted of isolated and phenol-purified full-length DNA of the Lyme21-9 clone labeled by random priming (Megaprime, Amersham) with [α-³²P]CTP (3000 Ci/mmol, Amersham). All blots were exposed to Kodak XAR film with intensifying screens at −70° C. for 4 days.

### EXAMPLE 8

### Cell Transfection

Intact plasmid DNA (10-25 μg) of the cDNA clone, Lyme21-9 (1), was transfected using the calcium phosphate precipitation method. Chinese hamster ovary cells (CHO-K1) were used to establish stable expression of the corresponding receptor, CMKRL1. The pCDₙₑₒ vector was co-transfected to allow for selection with G-418) Geneticin, 500 μg/ml). Single resistant colonies were isolated to obtain homogenous cell lines, and appropriate expression of the corresponding mRNA were verified by Northern blot hybridization with cDNA probes. SV40 transformed African green monkey kidney cells (COS-7) were used for transient expression. These cells were harvested after 48 hrs for preparation and isolation of membranes. Sham-transfected cells were used as controls throughout.

### EXAMPLE 9

### Production Of Monoclonal Antibodies (mAB)

A peptide was synthesized (Euro-Diagnostica, Sweden) corresponding to the first 15 of the N-terminal amino acid residues in CMKRL1, plus a C-terminal cysteine residue and then conjugated to BSA. Five mice were immunized i.p. 3 times at weekly intervals with peptide antigen mixed with the RIBI adjuvant system (Sigma). This was followed by a fourth injection 6 weeks afterwards. Ten days later the antibody response was checked in blood samples using ELISA (wells coated with peptide conjugated with KLH in bicarbonate buffer, pH 9.6) and indirect immunocytochemistry (CHO cells transfected with Lyme21-9, and sham-transfected controls). Mice with the strongest immune response were boosted by i.p. injection of the antigen mix and their spleen cells were used for fusion 3 days later. The spleen cells were fused with mouse myeloma SP 2/0 cells by the addition of PEG, followed by selection in HAT medium. Supernatants from wells with growing hybridomas were harvested and again tested by immunocytochemistry 9-20 days after fusion. Antibody-producing hybridomas were cloned out by limiting dilution.

### EXAMPLE 10

### Immunocytochemistry

CHO cells were grown on a microscope coverslips in 6-well culturing plates, and following removal of the medium and washing in PBS, fixed in methanol at 4° C. The cells were preincubated in 10% goat serum for 20 min, and the mouse mAb was added at varying concentrations, which was followed by a further incubation for 1 hr, all at RT. After thorough washing in PBS, the cells were incubated in FITC-labelled goat anti-mouse IgG (Jackson ImmunoResearch) for 1 hr at RT. After another rinsing, the coverslips with cells were mounted in glycerol on top of microscope slides and examined and photographed in a Leica Aristoplan fluorescence microscope equipped with appropriate excitation and barrier filters.

### EXAMPLE 11

### Intracellular Calcium Measurements

Transfected cells were seeded into 96-well plates (Corning-Costar) by dissociating confluent cultures using PBS containing 2 mM EDTA, diluting the resulting suspension 20x, and adding 50 μl per well. Following over-night incubation at 37° C., the growth medium was replaced with cold (40C) medium containing 1 μM Fura-42AM (Sigma) and 2.5 mM probenecid (Sigma), and the cells were incubated for one hour at 37° C. After 3 washes in extracellular medium buffer (135 mM NaCl, 4.6 mM KCl, 1.2 MM MgCl₂, 1.5 mM CaCl₂, 11 mM glucose, 11 mM Hepes, pH 7.4), the plates were mounted on the stage of a Nikon inverted fluorescence microscope attached to an SIT-camera and an image analysis system (Photon Technology International). Measurements were made using extracellular medium buffer at RT, and real-time digital background subtraction to compensate for the autofluorescence of the plastic plates.

Agonists were added in 50 pl of extracellular medium buffer and mixed thoroughly with the original contents of the well. ° Fluorescence images were obtained by alternate excitation at 340 Hand 380 nm, and the emitted light was measured at 510 nm. The software configuration allowed for storage of fluorescence information-from groups of cells, which could then be analyzed separately. Intracellular calcium concentrations were calibrated using the method described by William and Fay (5) and calculated from the aforementioned radiometric images using the mathematical relationship described by Grynkiewicz et al. (6).

For experiments involving calcium-free extracellular buffers, the buffer was prepared as described above, but without calcium and magnesium and with 10 mM EGTA. In addition to the preincubation with Fura-2, the cells were subsequently incubated a further 30 min in either normal extracellular buffer or the calcium-free buffer. Agonists were then added in normal buffer solutions or calcium-free buffer solutions, respectively. For experiments with the mAb, cells were first incubated with Fura-2 and then for an additional 30 min in medium containing the mAb, or normal growth medium, followed by 3 washes with extracellular buffer (to avoid problems created by serum protein autofluorescence).

### EXAMPLE 12

### Binding Assays

The transiently transfected COS-7 cells were washed with PBS, which was replaced with 50 mM TRIS/HCl buffer (pH 7.4) containing 1 mM EGTA and 5 MM MgCl₂, and scraped off. After homogenization for 20 sec in a Polytron homogenizer (Brinkmann, setting 5.5) the material was centrifuged at 20,000 rpm for 30 min at 4° C., followed by washing through homogenization in the TRIS binding buffer and subsequent recentrifugation, which was repeated twice. The pellet was finally resuspended by homogenization and stored in liquid nitrogen until used. For the binding studies, membrane material corresponding to 30 μg protein (Bradbury assay) was blended with ice-cold binding buffer (0.02 M Hepes buffer containing 10 mM CaCl₂ and MgCl₂) to a final volume of 70 μl and added to wells in a 96-well polypropylene plate. [³H]LTB₄ in varying concentrations and/or LTB₄ 1 μM were then added in an additional 70 μl of ice-cold binding buffer and mixed with the original well contents by briefly shaking the plate. The plate was then incubated at 4° C. for 1 hr before bound and free [³H]LTB₄ were separated by rapid filtration through Whatman glass fiber filters mounted in a cell harvester (Inotech). Each data point is the result of the mean of triplicate wells from three separate experiments, which have in turn been averaged.

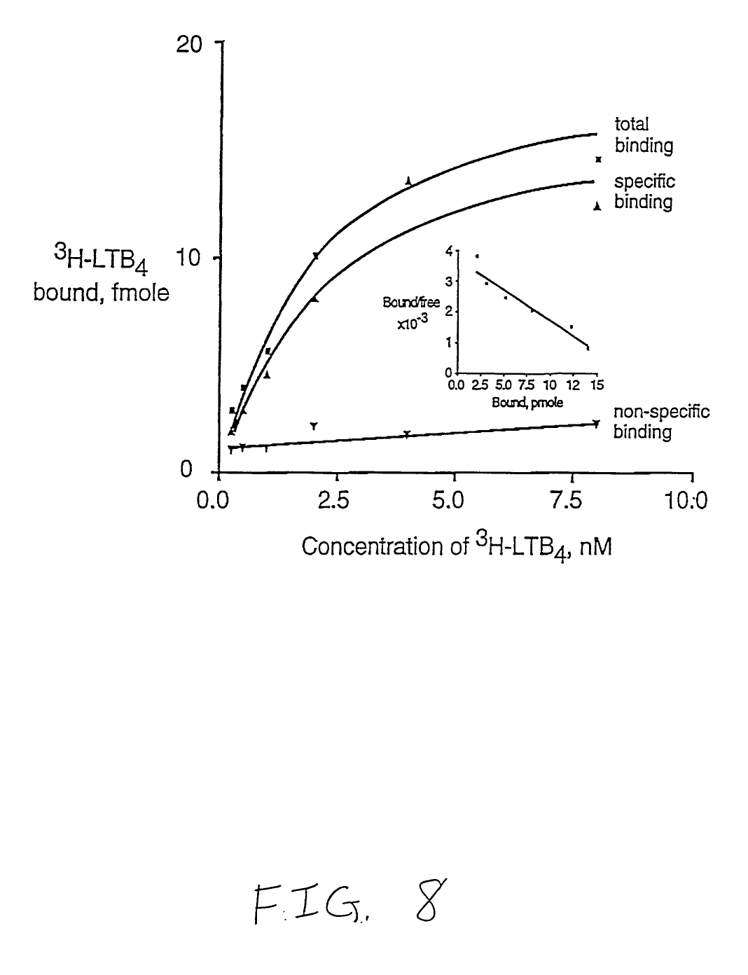

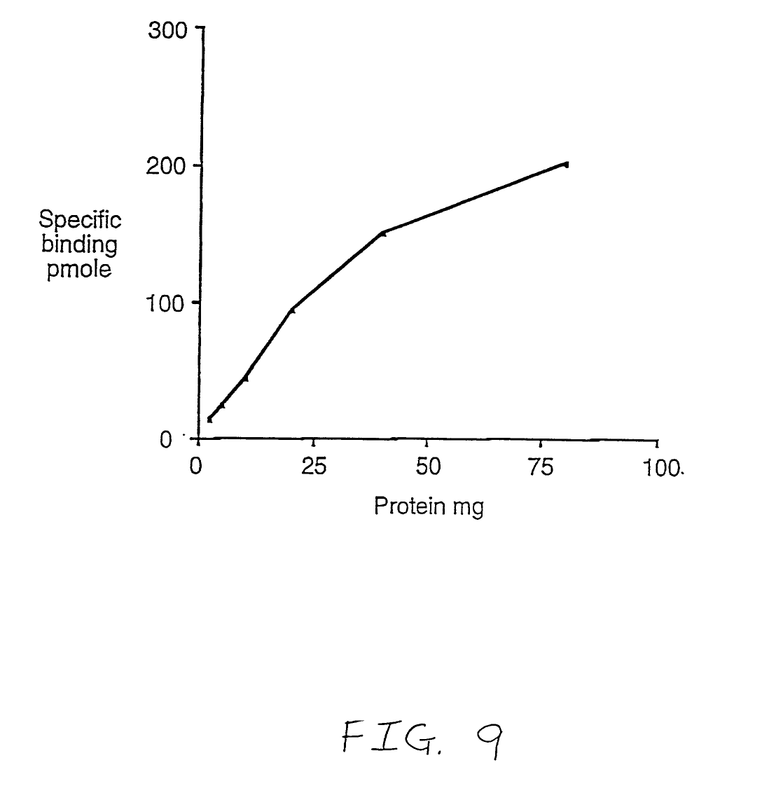

In the assays of binding of [³H]LTB₄ to isolate membrane preparations from COS-7 cells transfected with cDNA to express the receptor, CMKRL1, there was a concentration-dependent increase in both total and specific binding at 4° C. until saturation was reached (FIG. 8). Non-specific binding, i.e., the binding of tritiated ligand in the presence of 1 μM non-labelled ligand, represented less than 15% of the total binding and did not vary significantly with radioligand concentration. There was a near-linear increase in the specific binding of [³H]LTB₄ with increasing concentrations of membrane protein (FIG. 9), whereas membranes from sham-transfected cells exhibited no specific binding (data not shown). The saturation binding studies yielded a linear Scatchard plot shown as inset of FIG. 8. Thus, the radioligand identified a single class of high-affinity binding sites with an affinity constant (K_{d}) of 2.1+0.5 nM (mean ±SEM, n=3) and a Bₘₐₓ of 17.0+2.0 pmoles/mg protein.

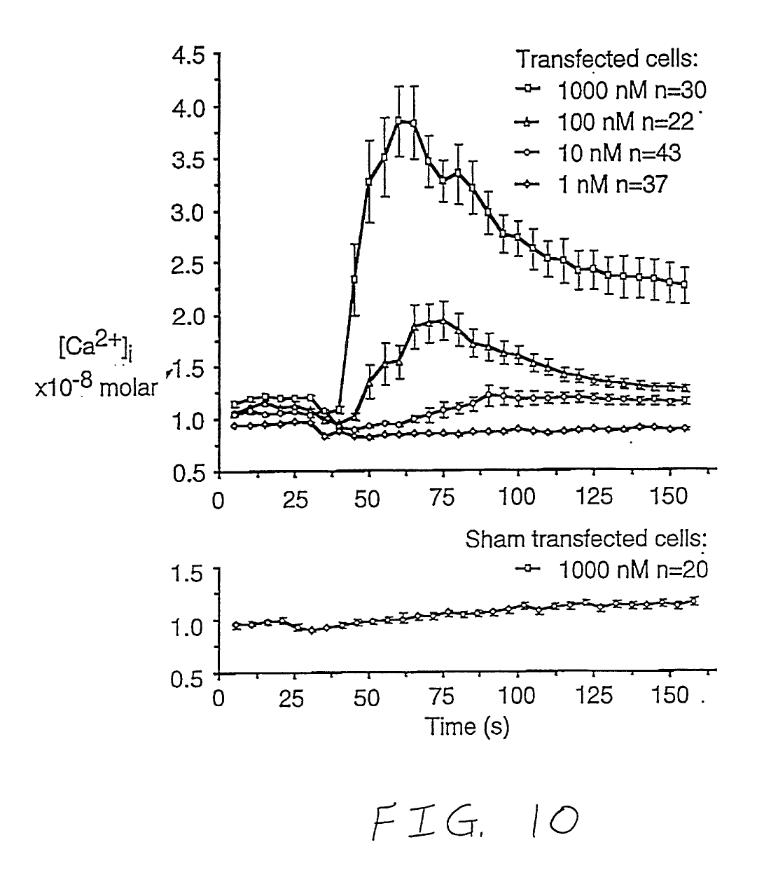

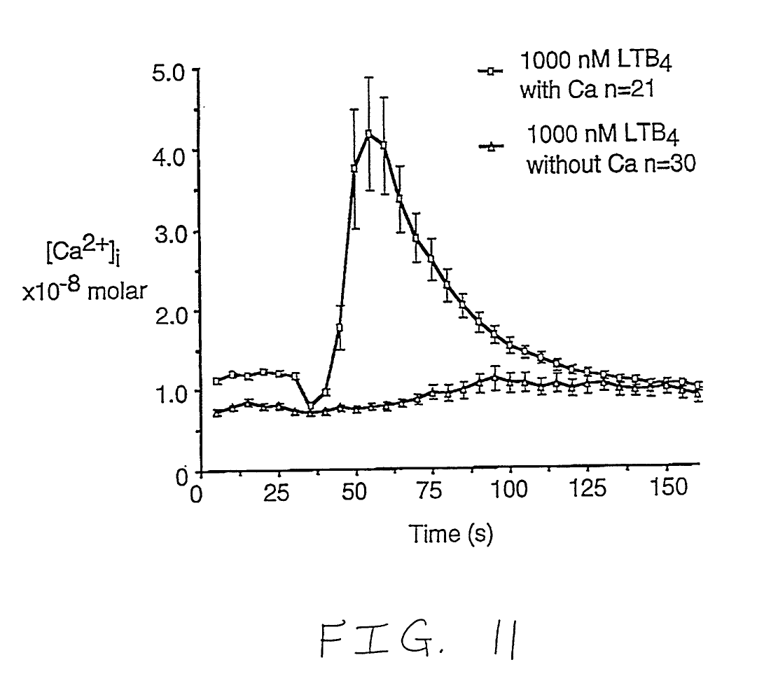

Addition of LTB₄ to CHO cells stably expressing CMKRL1 receptor message and preloaded with Fura-2 showed a rapid increase in the fluorescence ratio reflecting increased intracellular levels of calcium (FIG. 10). The effect was concentration-dependent between 1 and 1000 nM final concentrations in the test well, the onset being more rapid and the elevation steeper at the higher concentrations. After the maximum response had been attained the cellular calcium concentration slowly returned to baseline levels. LTB, was completely without effect at any dose level when added to sham-transfected control cells (shown for the 1000 nM concentration in FIG. 10, lower curve). When the regular cell medium was exchanged with calcium-free medium, LTB₄ no longer included any significant increase in intracellular calcium levels (FIG. 11).

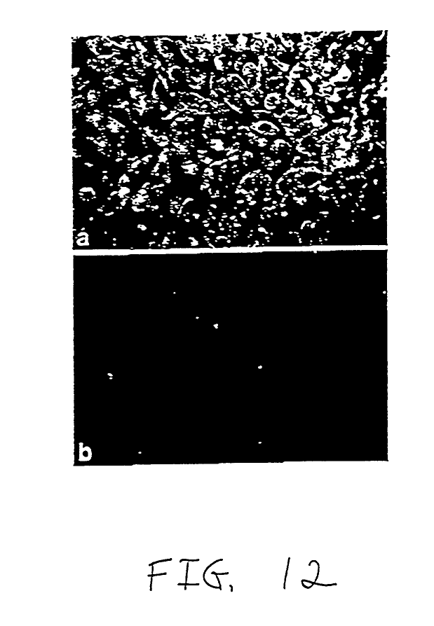

From a panel of 458 successfully growing hybridomas from mice immunized with a synthetic peptide corresponding to the N-terminus of CMKRL1, 48 supernatants were positive in the ELISA assay. Of these, 11 gave positive immunocytofluorescence reactions in transfected cells expressing CMKRL1, and following further cloning and growth of the individual hybridoma clones, 2 cell supernatants still showed positive immunoreactivity at the microscopic level. The immunocytochemical reaction of one mAb is illustrated in FIG. 12, which shows the distribution of a finely granular fluorescence in the periphery of CHO cells expressing CMKRL1 (FIG. 12a), whereas no fluorescence is seen in sham-transfected control cells (FIG. 12b). Also, no fluorescence was seen in control incubation with FITC-conjugated secondary antibody alone in either type of cell (not shown).

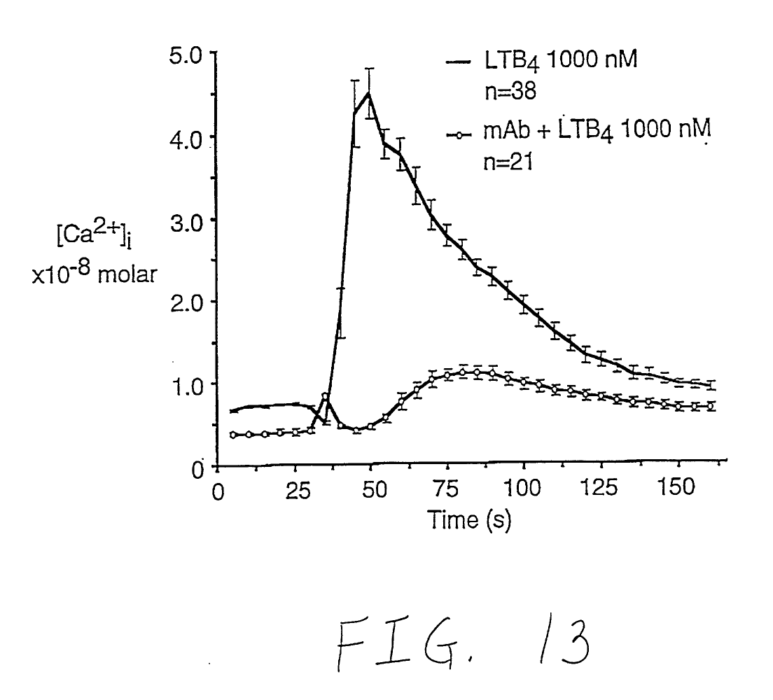

Preincubation of CMKRL1-expressing cells with the mAb illustration in FIG. 12 blocked the LTB₄-induced calcium response almost completely (FIG. 13).

In summary, this invention provides a new heptahelix receptor of the chemoattractant receptor family, which is capable of binding other ligands, including LTB4. The receptor and the polynucleotides encoding the receptor are useful indications of Burkitt's lymphoma because of its expression in such cells.

### REFERENCES

The following publications have been cited in the foregoing text and have been identified by a numeral in parentheses, each numeral corresponding to the respective publication in the following list. The entire disclosure of each of the following publications is relied upon and incorporated by reference herein.

Akbar, G. K. M., et al., J. Biol. Chem. 271:18363-18367 (1996). (2).

Alexander, S. P. H. & Peters, J. A., TiPS Receptor and Ion Channel Nomenclature Supplement, Elsevier Science, Amsterdam, (1997), pp. 50-51. (10).

Ahuja, S. K., Özcelik, T., Milatovitch, A., Francke, U., and Murphy, P. M. (1992). Molecular evolution of the human interleukin-8 receptor gene oluster. Nature Gen. 2: 31-36.

Birkenbach, M., Josefsen, K., Yalamanchili, R., Lenoir, G., and Kieff, E. (1993). Epstein-Barr virus-induced genes: First lymphocyte-specific G protein-coupled peptide receptors. J. Virol. 67: 2209-2220.

Bonner, T. I., Buckley, N. J., Young, A. C., and Brann, M. R. (1987). Identification of a family of muscarinic acetylcholine receptor genes. Science 237: 527-532.

Boulay, F., Tardif, M., Brouchon, L., and Vignais, P. (1990). Synthesis and use of a novel N-formyl peptide derivative to isolate a human N-formyl peptide receptor cDNA. Biochcem. Biophys. Res. Commun. 168: 1103-1109.

Brooker, G., Harper, J. F., Teraski, W. L., and Moylan, R. O. (1979). Radioimmunoassay of cyclic AMP and cyclic GMP. Adv. Cycl. Nucleotide Res. 10: 1-33.

Chen, C. and Okayama, H. (1987). High efficiency transformation of mammalian cells by plasmid DNA. Mol. Cell. Biol. 7: 2745-2752.

Chuntharapai. A., et al., J. Immunol. 152:1783-1789 (1994). (14).

Dixon, R. A. F., Kobilka, B. K., Strader, D. J., Benovic, J. L., Dohlman, H. G., Frielle, T., Bolanowski, M. A., Bennett, C. D., Rands, E., Diehl, R. E., Mumford, R. A., Slater, E. E., Sigal, I. S., Caron, M. G., Lefkowitz, R. J., and Strader, C. D. (1986). Cloning of the gene and cDNA for mammalian β-andrenergic receptor and homology with rhodopsin. Nature 321: 75-79.

Dobner, T., Wolf, I., Emrich, T., and Lipp. M. (1992). Differentiation-specific expression of a novel G protein, coupled receptor from Burkitt's lymphoma. Eur. J. Immunol. 22: 2795-2799.

Donath, P. D., et al., J. Exp. Med. 183:2437-2448 (1996). (15).

Feng, Y., Broder, C. C., Kennedy, P. E., and Berger, E. A. (1996). HIV-1 entry cofactor: Functional cDNA cloning of a seven-trans-membrane G protein-coupled receptor. Science 272: 872-876.

Forster, R., Emrich, T., Kremmer, E., and Lipp, M. (1994). Expression of the G-protein-coupled receptor BLR1 defines mature, recirculating B cells and a subset of T-helper memory cells. Blood 84: 830-840.

Gerard, N. P., and Gerard, C. (1991). The chemotactic receptor for human C5a anaphylatoxin. Nature 349: 614-617.

Giannini, E., et al., J. Biol. Chem. 270:19166-19172 (1996). (13).

Grynkiewicz, G., et al., J. Biol. Chem. 260:3440-3450 (1985). (6).

Hebert, T. E., et al., J. Biol. Chem. 271:16384-16392 (1996). (12).

Heng, H. H. Q., and Tsui, L. -C. (1993). Modes of DAPI banding and simultaneous in situ hybridization. Chromosoma 102: 325-332.

Heng, H. H. Q., and Tsui, L. -C. (1994). FISH detection on DAPI-banded chromosomes. In Methods in Molecular Biology: In Situ Hybridization Protocols” (K. H. A. Choo, Ed.), pp. 35-49. Humana Press, Clifton, N.J.

Herold, C. L., et al., Biochem. Biophys. Res. Commun. 235:717-721 (1997). (3).

Herzog, H., Hjort, Y. J., Shine, J., and Selbie, L. A. (1993). Molecular cloning, characterization, and localization of the human homolog to the reported bovine NPY Y3 receptor: Lack of NPY binding and activation. DNA Cell Biol. 12: 465-471.

Holmes, W. E., Lee, J., Kuang, W. -J., Rice, G. C., and Wood, W. I. (1991). Structure and functional expression of a human interleukin-8 receptor. Science 253: 1278-1280.

Kincade, P. W. (1994). B lymphopoiesis: Global factors, local control. Proc. Natl. Acad. Sci. USA 91: 2888-2889.

König, M., Mahan, L. C., Marsh, J. W., Fink, J. S. and Brownstein, M. J. (1991). Method for identifying ligands that bind to cloned Gₛ or Gᵢ-coupled receptors. Mol Cell. Neurosci. 2: 331-337.

Kozak, M. (1986). Point mutations define a sequence flanking the AUG initiator codon that modulates translation by eukaryotic ribosomes. Cell 44: 283-292.

Kubo, T., Fukuda, K., Mikami, A., Maeda, A., Takahashi, H., Mishina, M., Haga, T., Haga, K., Ichiyama, A., Kangawa, K., Kojima, M., Matsuo, H., Hirose, T., and Numa, S. (1986). Cloning, sequencing and expression of complementary DNA encoding the muscarinic acetylcholine receptor. Nature 323: 411-416.

Kunkel, S. L., Strieter, R. M., Lindley, I. J. D., and Westwick, J. (1995). Chemokines: New ligands, receptors and activities. Immunol. Today 16: 559-561.

Kyte, J., and Doolittle, R. F. (1982). A simple method for displaying the hydropathic character of a protein. J. Mol. Biol. 157: 105-132.

Libert, F., Parmentier, M., Lefort, A., Dinsart, C., Van Sande, J., Maenhaut, C., Simmons, M. J., Dumont, J. E., and Vassart, G. (1989). Selective amplification and cloning of four new members of the G protein-coupled receptor family. Science 244: 569-572.

Loetscher, M., Geiser, T., O'Reilly, T., Zwahlen, R., Baggiolini, M., and Moser, B. (1994). Cloning of a human seven-transmembrane domain receptor, LESTR, that is highly expressed in leukocytes. J. Biol. Chem. 269: 232-237.

Masu, Y., Nakayama, K., Tamaki, H., Harada, Y., Kuno, M., and Nakanishi, S. (1987). cDNA cloning of bovine substance-K receptor through oocyte expression system. Nature 329: 836-838.

Mattaj, I. W., and Seraphin, B. (1992). Picking the needle from the haystack. Curr. Biol. 2: 363-365.

Metters, K. M., J. Lipid Mediat. Cell Signal. 12:413-427 (1995). (9).

Morel, A., O'Carroll, A. -M., Brownstein, M. J., and Lolait, S. J. (1992). Molecular cloning and expression of a rat V1a arginine vasopressin receptor. Nature 356: 523-526.

Mullis, K. B., and Faloona, F. A. (1987). Specific synthesis ¹of DNA in vitro via a polymerase-catalyzed chain reaction. Methods Enzymol. 155: 335-350.

Murphy, P. M. (1994). The molecular biology of leukocyte chemoattractant receptors. Annu. Rev. Immunol. 12: 593-633.

Murphy, P.M., and McDermott, D. (1991). Functional expression of the human formyl peptide receptor in Xenopus oocytes requires a complementary human factor. J. Biol. Chem. 266: 12560-12567.

Ng, C. F., et al., J. Immunol. 147:3096-3103 (1991). (11).

Offermanns, S., and Simon, M. I., J. Biol. Chem. 270:15175-15180 (1995). (18).

Okayama, H., Kawaichi, M., Brownstein, M., Lee, F., Yokota, T., and Arai, K. (1987). High-efficiency cloning of full-length cDNA; construction and screening of cDNA expression libraries for mammalian cells. Methods Enzymol. 154: 3-28.

Owman, C., Blay, P., and Lolait, S. (1996). Cloning of Human cDNA Encoding a Novel Heptahelix Receptor Expressed in Burkitt's Lymphoma and Widely Distributed in Brain and Peripheral Tissues. Biochem. Biophys. Res. Comm. 228: 285-292.

Owman, C., Nilsson, C., and Lolait, S. (1996). Cloning of cDNA Encoding a Putative Chemoattractant Receptor. Genomics 37: 187-194.

Owman, C., et al., Genomics 37:187-194 (1996). (1).

Raport, C. J., Schweickart, V. L., Cantry, D., Eddy, R. L., Jr., Shows, T. B., Godiska, R., and Gray, P. W. (1996). New members of the chemokine receptor family. J. Leukocyte Biol. 59: 18-23. (7).

Raport, C. J., et al., J. Leukocyte Biol. 59:18-23 (1996).

Riabowol, K. T., Fink, J. S., Gilman, M. Z., Walsh, D. A., Goodman, R. H., and Feramisco, J. R. (1988). The catalytic subunit of cAMP-dependent protein kinase induces expression of genes containing cAMP-responsive enhancer elements. Nature 336: 83-86.

Riek, R. P., Handschumacher, M. D., Sung, S. -S., Tan, M., Glynias, M. J., Schluchter, M. D., Novotny, J., and Graham, R. M. (1995). Evolutionary conservation of both the hydrophilic and hydrophobic nature of transmembrane residues. J. Theor. Biol. 172: 245-258.

Rimland, J., Xin, W., Sweetnam, P., Saijoh, K., Nestler, E. J., and Duman, R. S. (1991). Sequence and expression of a neuropeptide Y receptor cDNA. Mol. Pharmacol. 40: 869-875.

Sambrook, J., Fritsch, E. F., and Maniatis, T. (1989). “Molecular Cloning. A Laboratory Manual,” Vol., pp. 1.42-1.52, Cold Spring Harbor Laboratory Press, Cold Spring Harbor, N.Y.

Samuelsson, B., TiPS 1:227-230 (1980). (8).

Schweickart, K. L., Raport, C. J., Godiska, R., Byers, M. G., Eddy, R. L., Shows, T. B., and Gray, P. W. (1994). Cloning of human and mouse EBIL, a lymphoid-specific G-protein-coupled receptor encoded on human chromosome 17q12-q21.2. Genomics 23: 643-650.

Siciliano, S. J., et al., Proc. Natl. Acad. Sci. USA 91:1214-1218 (1994). (16).

Spiegel, A. M., et al., Endocrinology Revs 13:536-565 (1992). (17).

Sugden, B. K., Marsh, K., and Yates, J. (1985). A vector that replicates as a plasmid and can be efficiently selected in B-lymphoblasts transformed by Epstein-Barr virus. Mol. Cell. Biol. 5: 410-413.

Thomas, K. M., Pyun, H. Y., and Navarro, J. (1990). Molecular cloning of the fMet-Leu-Phe receptor from neutrophils. J. Biol. Chem. 265: 20061-20064.

Watson, S., and Arkinstall, S. (1994). “The G-Protein Linked Receptor Facts Book, 95 pp. 1-427, Academic Press, London.

Williams, D. A. & Fay, F. S., Cell Calcium 11:75-83 (1990). (5).

Yokomizo, T., et al., Nature 387:620-624 (1997). (4).

What is claimed is:

1. A purified DNA molecule having the sequence:
CLONING OF NOVEL HUMAN CHEMOATTRACTANT RECEPTOR

| | | |
|---|---|---|
| −308 | acctgctacttgaaggccacacccagcc | −361 |
| −360 | tctcactcccttaccttcctcttcctctctcactgctccttcctggtctcttctcatctggccccacctctaaggcgtcctcctgccttctgggttgccctggaaaatagactacccccc | −241 |
| −240 | ctcctagtgaagggagtgggtaggggtttcagccccaccctcaggaagatgcgtcttccctgtcctctgctctgtggtacttcctctctggctgatttagcaaacagcacctagacctgg | −121 |
| −120 | gccaggcctttggcagtgggacagacccagggataggctacaccaccctgccctgaccctgggattggcatcagcttccaaccagttcctgccaaagcttgtaaggtcctcccgacggcc | −1 |
| | [symbol] I | |
| 1 | M X T T S S A A P P S L G V E F I S \overline{L L A I I L L S V A L A V G L P G S S T V V} | 40 |
| 1 | atgaacactacatcttctgcagcacccccctcactaggtgtagagttcatctctctgctggctatcatcctgctgtcagtggxgxtggctgtggggcttcccggcaacagctttgtggtg | 120 |
| | II | |
| 41 | \overline{W S} I L K R M Q K R S V T A \overline{L M V L M L A L A D L A V L L T A P F F L} N F L A Q | 80 |
| 121 | tggagtatcctgaaaaggatgcagaagcgctctgtcactgccctgatggtgctgaacctggccctggccgacctggccgtattgctcactgctccctttttccttcacttcctggcccaa | 240 |
| | III | |
| 81 | G T W S F G L A G C R L \overline{C M Y V C G V S M T A S V L L I T A K S L D} R S L A V A | 120 |
| 241 | ggcacctggagttttggactggctggctggttgccgcctgtgccactatgtctgcggagtcagcatgtacgccagtgtcctgcctatcacggtcatgagtctagaccgctcactggcggtggcc | |
| | IV | |
| 121 | A P F V S Q K L A T K A N A R R \overline{V L A G I M V L S P L L A T P V L A Y A T} V V P | 160 |
| 361 | cgcccctttgtgtcccagaagctacgcaccaaggcgatggcccggcgggtgctggcaggtatctgggtgttgtcctttctgctggccacacccgtcctcgcgtaccggacagtagtgccc | 480 |
| | [symbol] V | |
| 161 | W X T M M S L C F P R Y P S E G H R \overline{A F K L I F X A V T G F L L P F L A V V A S} | 200 |
| 481 | tggaaaacgaacatgagcctgtgcttcccgcggtaccccagcgaagggcaccgggccttccatctaatcttcgaggctgtcacgggcttcctgctgcccttcctggctgtggtggccagc | 600 |
| | VI | |
| 201 | Y S D I G R R L Q A R X R R F R R S R R T G R L \overline{V V L I I L T F A A F W L P Y M V V }240 | |
| 601 | tactcggacatagggcgccggctacaggcccggcgcttccgccgcagccgccgcaccggccgcctggtggtgctcatcatcctgaccttcgccgccttctggctgccctaccacgtggtg | 720 |
| | VII | |
| 241 | \overline{M L} A E A R R A L A C Q A A G L G L V G K R L S L A R X V L \overline{I A L A F L S S S V } | 280 |
| 721 | aacctggctgaggcccgccgcgcgctggccggccaggccgccgggttagggctcgtggggaagcggctgagcctggcccgtaacgtgcttatcgcactcgccttcctgagcagcagcgtg | 840 |
| 281 | \overline{W P V L Y A C A G G G} L L R S A G V G F V A K L L E G T G S K A S S T R R G G S | 320 |
| 841 | aaccccgtgctgtacgcgtgcgccggcggcggcctgctgcgtttggcgggcgtgggctttgtcgccaagctgctggagggcacgggctccgaggcgtccagcacgccgcgggggcagc | 960 |
| 321 | L G Q T A R S G P A A L X F G P S K S L T A S S F L K L N E L M | 352 |
| 961 | gctgggccagaccgctaggagcggccccgccgctctggagcccggcccttccgagagcctcactgcctccagccctctcaagttaaacgaactgaactaggcctggtggaaggaggcgcat | 1080 |
| 1081 | tttcctcctggcagaatgctagctctgagccagttcagtacctggaggagcaggggcgtggagggcgtggagggcgtgggagcgtgggaggcgggagtggagtggaagaagagggag | 1200 |
| 1201 | aggtggagcaaagtgagggccgagtgagagcgtgctccagcctggctcccacaggcagctttaaccattaaaactgaagtctga | 1283 |

SEQ ID NO:1.

2. A DNA molecule according to claim 1, which encodes a soluble human heptahelix receptor.

3. A DNA molecule according to claim 1 having the sequence: (I) in FIG. 1.

4. A DNA molecule according to claim 1 having the sequence: (II) in FIG. 1.

5. A DNA molecule according to claim 1 having the sequence: (III) in FIG. 1.

6. A DNA molecule according to claim 1 having the sequence: (IV) in FIG. 1.

7. A DNA molecule according to claim 1 having the sequence: (V) in FIG. 1.

8. A DNA molecule according to claim 1 having the sequence: (VI) in FIG. 1.

9. A DNA molecule according to claim 1 having the sequence: (VII) in FIG. 1.

10. A DNA sequence according to claim 1 encoding the heptahelix receptor polypeptide expressed by a microorganism selected from the group consisting of yeast, bacteria, and eukaryotic cells.

11. A recombinant vector comprising an expression vector containing a DNA molecule as claimed in any one of claims 1 to 10.

12. A host cell transduced or transfected with a vector as claimed in claim 12.

13. A method of making heptahelix receptor, which comprises providing a host cell comprising an expression vector containing the DNA molecule of claim 1, and expressing said DNA to produce said receptor.

14. A method according to claim 16, which comprises recovering the heptahelix receptor.

15. A method according to claim 16, wherein the host cell is a bacterium or yeast.

16. A heptahelix receptor having the sequence:

| | | |
|---|---|---|
| 1 | M N T T S S A A P P S L G V E F I S L L A I I L L S V A L A V G L P G N S F V V | 40 |
| 41 | W S I L k R M Q K R S V T A L M V L N L A L A D L A V L L T A P F F L H F L A Q | 80 |
| 81 | G T W S F G L A G C R L C H Y V C G V S M Y A S V L L I T A M S L D R S L A V A | 100 |
| 121 | R P F V S Q K L P T K A M A R R V L A G I W V L S F L L A T P V L A Y R T V V P | 160 |
| 161 | W K T N M S L C F P R Y P S E G H R A F H L I F E A V T G F L L P F L A V V A S | 200 |
| 201 | Y S D I G R R L Q A R R F R R S R R T G R L V V L I I L T F A A F W L P Y H V V | 240 |
| 241 | N L A E A R R A L A G Q A A G L G L V G K R L S L A R N V L I A L A F L S S S V | 280 |
| 281 | N P V L Y A C A G G G L L R S A G V G F V A K L L E G T G S E A S S T R R G G S | 320 |
| 321 | L G Q T A R S G P A A L E P G P S E S L T A S S P L K L N E L N (SEQ ID NO:2) | 352 |

17. A fragment of heptahelix receptor comprising up to about 100 consecutive amino acid residues in FIG. 1 and containing Asn-2.

18. A fragment of heptahelix receptor comprising up to about 100 consecutive amino acid residues in FIG. 1 and containing Asn-164.

19. A fragment of heptahelix receptor comprising up to about 200 consecutive amino acid residues in FIG. 1 and containing Cys-90 and Cys-168.

20. A fragment of heptahelix receptor selected from the group consisting of DNA molecules having the sequences (I), (II), (III), (IV), (V), (VI), and (VII) in FIG. 1.

21. A method of detecting Burkitt's lymphoma, wherein the method comprises providing disrupted human cells, contacting the cells with DNA as claimed in claim 1, and detecting a hybrid containing said DNA.

22. An antibody that specifically recognizes the heptahelix receptor as claimed in claim 16.

23. The antibody as claimed in claim 22, which is a monoclonal antibody.

24. A method of detecting Burkitt's lymphoma, wherein the method comprises providing human cells, contacting the cells with the antibody as claimed in claim 22, and detecting immunological complex containing said antibody.

25. A method for lowering the level of active leukotriene B4 in a mammal in need thereof, which comprises administering to said mammal a leukotriene B4-lowering amount of a receptor comprising the sequence of amino acids of SEQ ID NO:2.

26. A method for lowering the level of active leukotriene B4 in a mammal having inflammation, which comprises administering to said mammal a leukotriene B4-lowering amount of a leukotriene B4 receptor comprising the sequence of amino acids of SEQ ID NO:2.

27. A method for lowering the level of active leukotriene B4 in a mammal having bronchoconstriction, which comprises administering to said mammal a leukotriene B4-lowering amount of a leukotriene B4 receptor comprising the sequence of amino acids of SEQ ID NO:2.

28. A method for lowering the level of active leukotriene B4 in a mammal having arthritis, which comprises administering to said mammal a leukotriene B4-lowering amount of a leukotriene B4 receptor comprising the sequence of amino acids of SEQ ID NO:2.

29. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to inhibit activity of human leukotriene B4 on polymorphonuclear leukocytes or monocytes in said human to thereby inhibit said tissue injury.

30. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to modulate the inflammatory effect of leukotriene B4 on polymorphonuclear leukocytes or monocytes by counteracting cell movement induced by LTB₄ in said human.

31. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to inhibit the stimulatory effect of leukotriene B4 on adherence of polymorphonuclear leukocytes or monocytes in said human to thereby inhibit said tissue injury.

32. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to inhibit stimulatory effect of leukotriene B4 on oxidative burst of stimulated polymorphonuclear leukocytes in said human to thereby inhibit said tissue injury.

33. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to inhibit the stimulatory effect of leukotriene B4 on degranulation of stimulated polymorphonuclear leukocytes in said human to thereby inhibit said tissue injury.

34. A method of treating a human to inhibit tissue injury accompanying inflammation resulting from leukocyte activity induced by LTB₄ produced in response to an inflammatory stimulus in the human, wherein the method comprises administering to said human a leukotriene B₄ receptor comprising the sequence of amino acids of SEQ ID NO:2 in an amount sufficient to inhibit the effect of leukotriene B4 on oxidative burst or degranulation of stimulated neutrophils in said human to thereby inhibit said tissue injury.

35. A method for assaying a ligand or an antagonist or agonist for said ligand, wherein the method comprises:
(A) providing a heptahelix receptor as claimed in claim 16 or a fragment thereof comprising a binding domain for the ligand, antagonist, or agonist;
(B) incubating the receptor with a test sample suspected to contain the ligand, antagonist, or agonist; and
(C) detecting binding between the receptor and the ligand, antagonist, or agonist.

36. A method according to claim 35, wherein the receptor is in an external cell membrane of a host cell transfected or transduced with DNA encoding the receptor.

37. A method according to claim 35, wherein binding is detected by intracellular calcium level in the host cell.
